(19) United States
(12) Patent Application Publication
(10) Pub. No.: US 2025/0250529 A1
(43) Pub. Date: Aug. 7, 2025

(54) APPARATUS AND METHOD FOR AUTOMATED ANALYSIS OF MICROBIOLOGICAL SAMPLES

(71) Applicant: Spectacular Labs, Inc., Richmond, CA (US)

(72) Inventors: Okhtay Montazeri, Richmond, CA (US); Leopoldo T. Saavedra, San Francisco, CA (US)

(73) Assignee: Spectacular Labs, Inc., Richmond, CA (US)

(21) Appl. No.: 19/046,493

(22) Filed: Feb. 5, 2025

Related U.S. Application Data
(60) Provisional application No. 63/550,595, filed on Feb. 6, 2024.

(51) Int. Cl. C12M 1/34; C12M 1/00; C12M 1/12; C12M 3/00; C12M 3/06

(52) U.S. Cl. CPC C12M 41/46; C12M 23/16; C12M 23/26; C12M 23/38; C12M 23/40; C12M 23/42; C12M 23/44; C12M 25/02; C12M 37/00; C12M 41/12

(57) ABSTRACT

A microbiological analysis module and analysis station is disclosed. The microbiological analysis module comprises a culture enrichment chamber subassembly, insertable within an attachment collar. The attachment collar is attachable to a cartridge adapter. A microfluidic cartridge subassembly comprising a stage extending laterally from a receptacle. The stage comprises a channel manifold having one or more channels fluidically coupled to the culture enrichment chamber and to one or more analysis chambers, a prime chamber and reagent chambers. The chambers are integral with the channel manifold.

### CLAIM FOR PRIORITY

This application claims priority to U.S. Provisional Patent Application No. 63/550,595, filed on Feb. 6, 2024, titled “APPARATUS AND METHOD FOR AUTOMATED ANALYSIS OF MICROBIOLOGICAL SAMPLES,” which is incorporated by reference in its entirety for all purposes. This application is hereby incorporated by reference in its entirety for all purposes.

### BACKGROUND

The ability to rapidly detect pathogens and other microbes at food preparation facilities, such as consumer product manufacturers and food processing plants, is paramount for the public health while ensuring timely distribution of products. To date, some important bacteriological and viral assays require culturing to achieve sufficient confidence in detection and identification of pathogens. Culturing microbes may generally take 24 to 72 hours or longer. In many situations, samples are sent to an off-site bacteriological laboratory which requires additional transportation and wait times. Therefore, the total turn-around time from sampling to results may take several days, in practice. In some cases where the initial microorganism load is assumed to be relatively high, rapid assay analyzers which are commercially available, may be used for use in on-site industrial laboratories. These rapid assay analyzers do not require culturing as the initial load is assumed to be sufficiently high. The assays themselves may take minutes to hours to complete. Most rapid assay analyzers may be relatively small and modular units that employ automated or robotic sample preparation and detection. They may be based on enzyme-linked immunosorbent assay (ELISA) protocols or polymerase chain reaction (PCR) protocols. While these rapid apparatuses might serve their purpose, without an initial culturing or enrichment they have a limited set of use cases. Enrichment is needed to ensure that the sample is safe, not just for immediate consumption, but for the duration of its expected shelf-life, which may be days, weeks, or months in the future. Specifically, an initial enrichment is required to ensure that a) the microorganisms in question are indeed viable and not dead, and b) very low initial microorganism levels are amplified to make their detection reliable and reproducible. As such, combining rapid testing with enrichment in a smart and easy to use system can help prevent many outbreaks that currently go undetected. Such an approach offers a significant improvement over currently available rapid test systems that only rely on DNA fragments for pathogen identification, and do not test for microorganism viability. These rapid test systems do not include an enrichment step, and as a result are relatively insensitive to microorganism- viability resulting in high false positives. Other systems requiring prolonged incubation periods without integrating incubation and rapid testing may result in delaying timely reporting of results.

### BRIEF DESCRIPTION OF THE DRAWINGS

The embodiments of the disclosure will be understood more fully from the detailed description given below and from the accompanying drawings of various embodiments of the disclosure, which, however, should not be taken to limit the disclosure to the specific embodiments, but are for explanation and understanding only.

FIG. 1A illustrates a cross-sectional view of an analysis assembly, in accordance with at least one embodiment.

FIG. 1B illustrates an isometric view of a microfluidic assembly, in accordance with at least one embodiment.

FIG. 1C illustrates a perspective view of a microfluidic assembly, showing lower portions of a receptacle and a platform, in accordance with at least one embodiment.

FIG. 1D illustrates an exploded view of microfluidic assembly, in accordance with at least one embodiment.

FIG. 1E illustrates a perspective view of a rotatable selector valve assembly engaged with a slotted cap, in accordance with at least one embodiment.

FIG. 1F illustrates a cross-sectional view of a rotatable selector valve assembly, in accordance with at least one embodiment.

FIG. 2A illustrates a 3D view of self-contained microbiological analysis cartridge (SCMAC) assembly, in accordance with at least one embodiment.

FIG. 2B illustrates an exploded view 3D of a SCMAC cartridge assembly, in accordance with at least one embodiment.

FIG. 3A illustrates a 3D isometric view of the SCMAC cartridge assembly, in accordance with at least one embodiment.

FIG. 3B illustrates a 3D view of a channel manifold, in accordance with at least one embodiment.

FIG. 3C illustrates a reverse side of the channel manifold, in accordance with at least one embodiment.

FIG. 4 illustrates a light guide assembly, comprising light guides integral with an optical window, in accordance with at least one embodiment.

FIG. 5A illustrates a first perspective view of an analysis station, in accordance with at least one embodiment.

FIG. 5B illustrates a second perspective view of a portion of an analysis station, in accordance with at least one embodiment.

FIG. 5C illustrates a third perspective view showing an enlarged view of an analysis station, in accordance with at least one embodiment.

FIG. 5D illustrates a fourth perspective view of an analysis station, in accordance with at least one embodiment.

FIG. 5E illustrates a perspective view of an enclosure for the analysis station, in accordance with at least one embodiment.

FIG. 6 illustrates a flow chart summarizing an exemplary method for an operating analysis station, in accordance with at least one embodiment.

### DETAILED DESCRIPTION

Disclosed herein is a self-contained sample preparation and analysis laboratory system comprising an integrated laboratory comprising a culture enrichment chamber subassembly and a microfluidic cartridge subassembly. In at least one embodiment, the microfluidic subassembly is attachable to the culture enrichment chamber once a microbiological sample is inoculated or immersed (e.g., a swab or wipe) into a liquid microbiological growth medium within the fluid container. A microbiological sample may be a liquid or solid object that may harbor microorganisms. In at least one embodiment, the microfluidic assembly comprises a microfluidic cartridge integrally attached to a receptacle for the fluid container. In at least one embodiment, the microfluidic cartridge extends laterally from the receptacle, supported by a cantilevered platform extending from a sidewall of the receptacle. In at least one embodiment, the microfluidic cartridge is in fluidic communication with the interior of the receptacle and attached fluid container.

In at least one embodiment, the microfluidic cartridge subassembly comprises one or more analysis chambers that are mechanically integral with the structure of the microfluidic cartridge. In at least one embodiment, the one or more analysis chambers communicate fluidically with one or more channels on a microfluidic manifold, which is part of the microfluidic cartridge. For example, the one or more analysis chambers extend below the cartridge orthogonally to the plane of the cartridge. In at least one embodiment, the one or more analysis chambers can have the shape of a test tube or centrifuge tube. In at least one embodiment, the microfluidic cartridge subassembly is configured to perform biochemical analyses on microbiological samples within the one or more analysis chambers. In at least one embodiment, the one or more analysis chambers may be prefilled with reagent solution or a dry powder or tablet. In at least one embodiment, one or more reagent vessels may be included on the microfluidic manifold. For example, a reagent chamber may contain a lysis medium for lysing microorganism cells. The lysis medium may be transferred to a first analysis chamber to lyse cells or viral particles from a sample that is also transferred into the first analysis chamber. A second analysis chamber may contain a solution comprising a diagnostic medium for reacting with biomolecules extracted from the lysed cells to which an aliquot of lysed solution is transferred.

In at least one embodiment, the self-contained sample preparation and analysis laboratory system further comprises an automated analytical base station into which the culture enrichment chamber and microfluidic cartridge subassembly is inserted for analyzing microbiological samples cultured within the culture enrichment chamber.

In at least one embodiment, the microfluidic cartridge assembly comprises a receptacle from which the microfluidic manifold extends. In at least one embodiment, the receptacle has a cavity surrounded by a sidewall to which the microfluidic manifold is cantilevered. In at least one embodiment, the cavity and sidewall have cylindrical symmetry. In at least one embodiment, the receptacle comprises a floor and an open top. In at least one embodiment, the receptacle is configured to communicate fluidically with the microfluidic manifold. In at least one embodiment, the receptacle is configured for attachment of the culture enrichment chamber. In at least one embodiment, the culture enrichment chamber has a non-rigid wall comprising a flexible material that enables expansion and collapse of the culture enrichment chamber. The culture enrichment chamber may be attached to the receptacle by a rigid cartridge adapter.

In at least one embodiment, an opening in the floor of the receptacle is in fluidic communication with the microfluidic manifold. In at least one embodiment, the microfluidic manifold comprises one or more channels (e.g., microchannels) on a platform, whereby the one or more channels may be configured for fluid transfer. In at least one embodiment, the form factor of the microfluidic manifold may be substantially rectangular or trapezoidal. In at least one embodiment, side panels extend below the edges of the microfluidic manifold, forming a rigid skirt.

In at least one embodiment, the microfluidic cartridge assembly may comprise an upper enclosure above the microfluidic manifold. In at least one embodiment, the upper enclosure comprises an opening through which a portion of a rotatable selector valve shaft extends. In at least one embodiment, the rotatable selector valve shaft may have a tubular form factor, whereby the rotatable selector valve shaft comprises a hollow interior. In at least one embodiment, the hollow interior is configured to accommodate a compliant plunger tip that is movable within the hollow interior of the shaft. As will be described below, the hollow interior of the shaft is accessible through an opening at the top of the shaft. The opening is configured to enable an extensible plunger shaft to enter within the hollow interior from above the platform, where the extensible plunger shaft is actuated by a motor that may engage the plunger tip.

In at least one embodiment, the extensible plunger shaft may move the plunger tip along the length of the hollow interior of the shaft to create a pumping action. The pumping action may enable the rotatable selector valve to move fluids within channels on the microfluidic manifold. A valve handle may protrude through an opening in the upper enclosure to engage with a motor shaft to enable rotation of the rotatable selector valve. The rotation enables selection of different channels for fluid transfer. In at least one embodiment, a lower end of the rotatable selector valve shaft is coupled to a rotatable disk. In at least one embodiment, the rotatable disk comprises an opening on its lower surface. In at least one embodiment, the lower surface of the rotatable disk is seated on the microfluidic manifold at the center of a circular configuration of channel termini. In at least one embodiment, the shaft and disk may be rotated such that the opening at the bottom of the disk aligns with any of the channels. Such an alignment provides a portal for fluid to flow between the individual channel and the hollow interior of the rotatable selector valve shaft to enable fluid transfer to or from the channel by pipetting action.

Each of the channels on the microfluidic manifold extends between a first terminal accessible to a rotatable selector valve and a second terminal. The second terminal may be in fluidic communication with any one of the one or more analysis chambers, reagent chambers, and the receptacle cavity floor access opening. In at least one embodiment, a prime chamber is included for storage of a sample of microbiological sample solution transferred from the culture enrichment chamber. One channel of the microfluidic manifold may extend from the rotatable selector valve and the prime chamber. The contents of the prime chamber may provide access to sample enrichment fluid to a user by enabling manual withdrawal of such fluid by syringe, for example. In this way, the culture enrichment fluid can be made available for external assays, such as species identification verification. In at least one embodiment, the upper enclosure comprises an opening over the prime chamber for insertion of a syringe needle into the prime chamber for withdrawal of culture enrichment fluid by use of an external syringe.

In at least one embodiment, the fluid transfer to individual analysis chambers may be actuated by pipetting action caused by the motion of the plunger tip within the hollow interior of the rotatable shaft. For example, fluid may be withdrawn into the hollow interior by upward motion of the plunger tip within the rotatable shaft, whereas fluid may be forced from the hollow interior into a channel coupled to a analysis chamber by downward motion of the plunger tip. In at least one embodiment, the plunger tip is moved by a plunger shaft that may be actuated by a motor exterior to the assembly. The displaced volume of fluid by the plunger shaft may be controlled by precision linear motion of the plunger tip within the hollow interior volume of the rotatable shaft.

As noted above, the culture enrichment chamber may comprise a compliant sac that is insertable into a more rigid holder that attaches by means of a cartridge adapter to the receptacle. The holder may serve to, for example, carry the compliant culture enrichment chamber from a lab bench to attach to the cartridge subassembly through the cartridge adapter. The cartridge adapter facilities engagement and attachment of the culture enrichment chamber to the cartridge assembly. In at least one embodiment, the culture enrichment chamber comprises a lip about its mouth, whereby the lip is configured to rest on the rim of the cartridge adapter when inserted therein.

The subassembly comprising the holder, cartridge adapter and the culture enrichment chamber may then be attached to the receptacle by engagement of mating threads, for example. Pairs of locking tabs may also be included to lock parts together after a predetermined torque is attained. For example, rotation of the holder relative to the cartridge adapter is limited by the position of the locking tabs and the thread design, whereby a predetermined torque is applied on the lip of the culture enrichment chamber. The predetermined torque may provide adequate force to enable hermetic sealing of the culture enrichment chamber when inverted. The receptacle functions as a cap hermetically sealing the compliant culture enrichment chamber so that leakage and contamination may be avoided. The rigid holder may provide a protective shield over the more delicate culture enrichment chamber insert. Subsequent expansion or contraction of the compliant insert during incubation and fluid transfer may occur, and by allowing pressure within the compliant culture enrichment chamber to rapidly equalize via the expansion, pressure within the culture enrichment chamber may be kept low enough so as not to leak through the seal at the lip.

In at least one embodiment, the compliant culture enrichment chamber may be presterilized and pre-filled with a sterile microbial growth medium and stored in a sterile package prior to use. In at least one embodiment, multiple sterile culture enrichment chambers may be stored empty in a compact format, and sterile growth medium may be added immediately prior to sample inoculation. Samples containing microorganisms or suspected of harboring pathogens, for example, may be inoculated or placed in some manner within the sterile growth medium contained by the culture enrichment chambers.

In at least one embodiment, self-contained microbiological analysis module (SCMAM) assembly comprising the microfluidic cartridge and culture enrichment chamber may be engaged with an autonomously operated analytical station instrument. The autonomous analysis station instrument may enable conduction of biochemical assays, such as polymerase chain reaction to indicate presence and/or identification of microorganisms carried by the sample via biochemical reactions occurring within the analysis chambers integrated on the microfluidic cartridge. Selected biochemical reactions may generate indicator analytes specific to that can be detected, for example by optical components configured to measure fluorescence, absorption, or by electrochemical sensor detection of analytes within analytical base station instrument.

In at least one embodiment, the analytical base station instrument comprises an enclosure surrounding a chassis to which various electronic and mechanical components may be attached. In at least one embodiment, the analytical base station instrument comprises a vertical mounting column comprising a plurality of bolt holes for aligned attachment of some components. The vertical mounting column may be affixed to the chassis by bolts or other fasteners, for example. In at least one embodiment, a stage for placement of the fluid container assembly may be affixed to the vertical mount. In at least one embodiment, one or more heater/optical blocks for aiding docking of the microfluidic cartridge (attached to the receptacle) within the analytical base station. The heater/optical blocks have openings for receiving the one or more analysis chambers and optionally the reagent chamber. The heater/optical blocks may contain LEDs or portals to pass light from LEDs through the analysis chambers. Such LEDs may serve as light sources for providing appropriate wavelengths of light for optical assays. In at least one embodiment, light guides as transparent rods extending from the top into the interior of the analysis chambers, are included for guiding light vertically to an optical window provided above the one or more analysis chambers. The light guides are optional but may increase optical efficiency by guiding most of the light, such as light from fluorescence to photodetectors positioned at the top of the reaction vessels. The optical window may be accessible to photodetectors aligned with optical window apertures in the upper enclosure, which are aligned with the tops of the analysis chambers. As an example of how the light guides and optical window are configured to aid in optical assays, fluorescent light emitted by analytes excited by LEDs in the instrument may be detected by this arrangement. In at least one embodiment, the heater/optical blocks may be ramped. The ramped portion of the heater/optical blocks may be oriented at an oblique angle, forming a ramp configured to guide the alignment of the analysis chambers.

For example, when engaging a fluid container assembly into the analytical base station, the microfluidic cartridge may be inserted first, and by pushing the assembly forward, analysis chambers extending below the channel manifold within the microfluidic cartridge encounter the ramped portion of the heater/optical blocks. As the analysis chambers ride up the ramps, the analysis chambers reach the flat top of the ramps and fall into openings for engagement of the analysis chambers. At this point, the microfluidic cartridge is engaged. The receptacle portion of the SCMAM assembly may be seated on a stage. In at least one embodiment, the stage is attached to the chassis or to a lower shelf affixed to the mounting column of the instrument. The bottom surface of the receptacle may also slide along the stage upon which it is centered when the analysis chambers are dropped into the openings in the ramps. Upon engagement of the analysis chambers with the openings on top of the ramps, the entire assembly is aligned within the analytical base station. In at least one embodiment, the analysis chambers are engaged within chambers that extend below the openings. Such engagement may lock in place the alignment of the fluid container assembly.

In at least one embodiment, an upper rim of the rotatable shaft of the rotatable valve may protrude through an opening on the upper panel of the platform. In at least one embodiment, the upper rim of the rotatable shaft may not extend over the upper panel but may be exposed. In at least one embodiment, the upper rim may be slotted to engage a motor shaft of a rotatable motor that is mounted within the analytical base station. For example, the rotatable motor may be mounted on the mounting column via a bracket.

In at least one embodiment, a slotted cap may be included in the platform subassembly. In at least one embodiment, the slotted cap may be configured to engage a collar on the motor shaft. The collar may comprise a blade that engages the slot in the slotted cap, providing a means to permit the platform to slide into place without raising and lowering the motor shaft relative to the platform during engagement of the latter. For example, the blade and slot may be oriented such that the blade may slide into the slot during engagement of the fluid container assembly with the analytical base station.

In at least one embodiment, analysis chamber chambers are cavities within heating blocks that are contained inside hollow spaced within the ramps, whereby the cavities may have approximate sizes and shapes of the analysis chambers. For example, the ramps may be shells comprising hollow interiors. In at least one embodiment, analysis chambers may have light inlet openings bored into a side of the analysis chambers and into the bottom of the analysis chambers. In at least one embodiment, light inlet openings may serve to illuminate analysis chambers by a light source, such as a laser from the side, whereby the light entering the analysis chamber from the side may excite fluorescence of one or more analytes. Fluorescent light from the analyte may propagate in orthogonal directions relative to the direction of the excitation. An opening at the bottom of the chamber may enable fluorescent light to be detected out of the path of the excitation light.

In at least one embodiment, the outer container may be a standard or non-standard bacteriological culture tube, centrifuge tube, or cup made from a non-compliant (e.g., semirigid) plastic material, such as polypropylene, that may have a volumetric capacity ranging from 15 milliliters (ml) to 1000 ml. In at least one embodiment, the fluid container may be prefilled with a liquid culture medium for culturing microorganisms or eukaryotic cells. In other embodiments, the fluidic container may contain a medium for dilution or preconditioning of non-biological samples, such as water samples or other liquid samples.

In at least one embodiment, the self-contained sample preparation and analysis laboratory system comprises an analysis base station onto which the sample preparation assembly may dock. In at least one embodiment, the disclosed sample preparation lid may be configured to fit standard or non-standard tubular and non-tubular fluid containers, such as culture tubes, centrifuge tubes, test tubes, culture bottles, jars, and the like. In at least one embodiment, the fluid container may be entirely straight walled, entirely conical, or a hybrid of straight walls and conical tips. In at least one embodiment, the disclosed sample preparation lid may replace standard screw caps or insertion caps that are commonly employed to fit such fluid containers.

In at least one embodiment, the SCMAM assembly may comprise substantially the same polymeric materials. In at least one embodiment, sample preparation analysis chambers may comprise polymer formulations that provide flexibility. In at least one embodiment, the lid body may comprise a polymer material that provides strength. In at least one embodiment, the lid body and sample preparation analysis chambers may comprise high-density polyethylene or polypropylene. In at least one embodiment, the sample preparation lid assembly (including lid body and sample preparation analysis chambers) may be fabricated by injection molding, 3D printing, or other suitable additive processes. In at least one embodiment, the sample preparation analysis chambers and lid bodies may be manufactured in separate processes. In at least one embodiment, separately fabricated sample preparation analysis chambers and lid bodies may be assembled in a subsequent assembly process. In at least one embodiment, sample preparation analysis chambers may be formed integrally with the lid body in a single or multi-step injection molding or 3D printing process.

In at least one embodiment, the automated analysis station may effectuate and manage fluid transfers between the fluid container and the microfluidic cartridge. In at least one embodiment, the automated analysis station may also manage fluid transfers between the one or more analysis chambers on the microfluidic cartridge. In at least one embodiment, the automated analysis station comprises temperature-controlled chambers into which the analysis chambers may be inserted. In at least one embodiment, automated analysis station may be engaged by manual or automated insertion of the sample preparation analysis chambers into corresponding chambers. As an example, in at least one embodiment, two sample preparation analysis chambers may be simultaneously inserted into a first and second chamber, respectively.

In this disclosure, it is understood that the terms “over,” “under,” “above,” “below,” “upper,” “lower,” “top,” and “bottom” have the usual structural meanings, referring to relative vertical positions within structural embodiments and to their immediate environment as viewed within the associated figures. Similarly, the terms “left,” “right,” “side,” and “sideways” have the usual structural meanings, referring to relative horizontal positions within structural embodiments and within their immediate environment as viewed within the associated figures.

The terms “substantial” or “substantially” are used within this disclosure to mean “the greater part of,” “mostly,” or “mostly to fully.” For example, “substantially” may qualitatively indicate a measure within 10% of a quantifiable attribute, with the possibility that the measure may range from 90% to 100% of the quantifiable attribute.

Views labeled “cross-sectional,” “profile,” “plan,” and “isometric” correspond to orthogonal planes within a cartesian coordinate system. Thus, cross-sectional and profile views are taken in the x-z plane, plan views are taken in the x-y plane, and isometric views are taken in a 3-dimensional cartesian coordinate system (x-y-z). Where appropriate, drawings are labeled with axes to indicate the orientation of the figure.

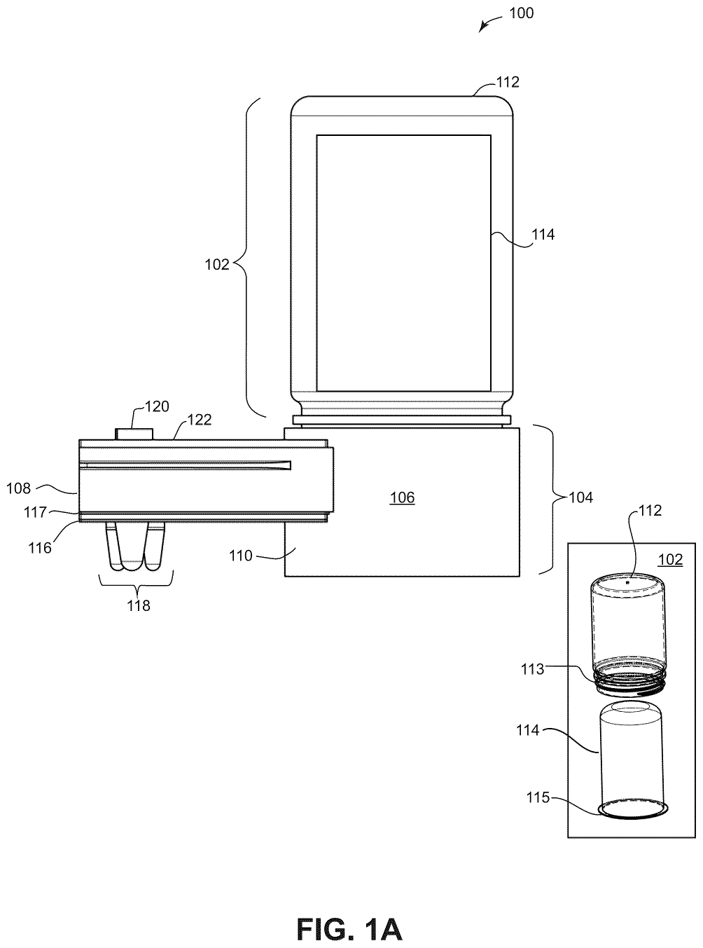

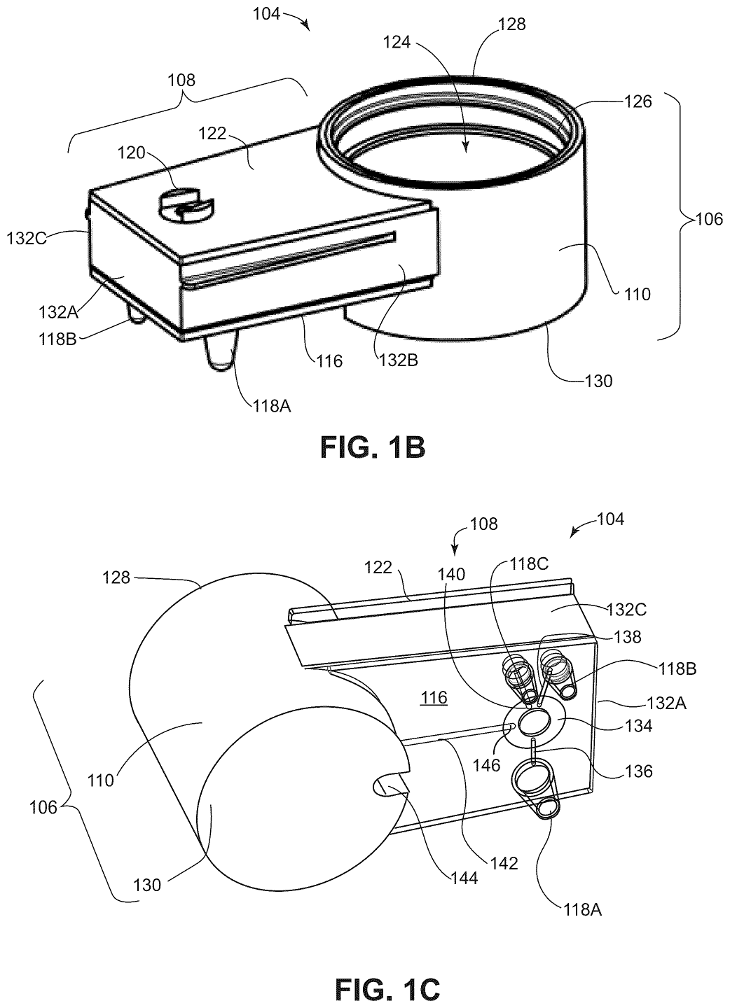

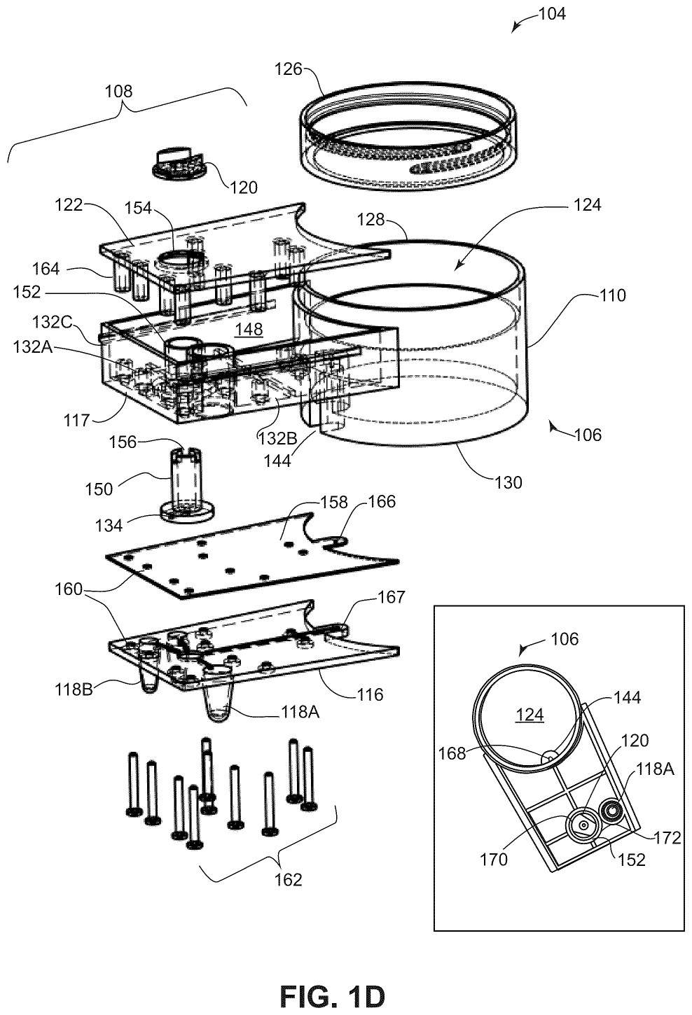

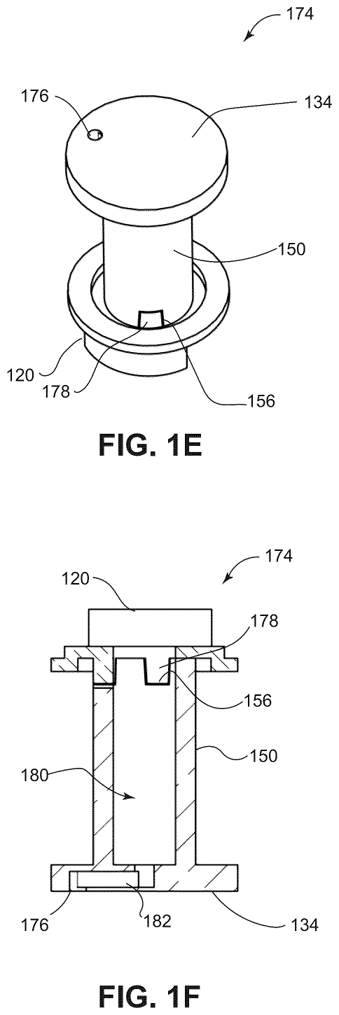

FIG. 1A illustrates a profile view of self-contained microbiological analysis module (SCMAM) assembly 100, in accordance with at least one embodiment. In at least one embodiment, SCMAM assembly 100 comprises culture enrichment chamber subassembly 102, with which microfluidic cartridge subassembly 104 may be attached. In at least one embodiment, microfluidic cartridge subassembly 104 comprises receptacle 106 and microfluidic cartridge subassembly 108 extending from sidewall 110 of receptacle 106. In at least one embodiment, culture enrichment chamber subassembly 102 comprises outer container 112, which is a tubular vessel, such as a culture tube or bottle. In at least one embodiment, a compliant insert 114 is contained within outer container 112. The inset in the figure shows an exploded view of culture enrichment chamber subassembly 102, showing threaded portion 113 of outer container 112. In at least one embodiment, compliant insert 114 comprises a lip 115 on its rim. In at least one embodiment, lip 115 is configured to seat under threaded portion 113 of outer container so that compliant insert 114 seals when assembled to microfluidic subassembly 104.

In at least one embodiment, outer container 112 comprises a non-compliant (e.g., semirigid) plastic material, such as but not limited to, polypropylene, high-density polyethylene (HDPE), perfluoroalkoxy polymer (PFA), or polytetrafluoroethylene (PTFE). In at least one embodiment, compliant insert 114 comprises a suitable flexible polymer, such as polydimethylsiloxane (PDMS), polypropylene, or HDPE. In at least one embodiment, the polymer may have a suitable range of elasticity to enable deformation of insert 114 by small changes in pressure. In at least one embodiment, sample preparation analysis chambers may comprise a suitable polymer material, such as polydimethylsiloxane (PDMS), or alternatively, high-density polyethylene. In at least one embodiment, receptacle 106 and microfluidic cartridge subassembly 108 may also comprise high-density polyethylene, or a rigid thermoset plastic, such as acrylonitrile butadiene styrene (ABS).

In at least one embodiment, microfluidic cartridge subassembly 108 comprises channel manifold 116. In at least one embodiment, one or more analysis chambers 118 extend orthogonally from channel manifold 116. In at least one embodiment, channel manifold 116 may be unitary with analysis chambers 118. In at least one embodiment, channel manifold 116 and analysis chambers 118 may be manufactured in a single additive process, such as injection molding or 3D printing. In at least one embodiment, channel manifold 116 may be manufactured separately from analysis chambers 118. For example, channel manifold 116 may be made by a first injection molding process. In at least one embodiment, analysis chambers 118 may be made in a second injection molding process. In at least one embodiment, analysis chambers 118 may be assembled onto channel manifold 116 in a subsequent manufacturing step.

In at least one embodiment, microfluidic subassembly 104 may be a molded structure comprising a rigid polymer, such as ABS. In at least one embodiment, microfluidic cartridge subassembly 108 is integral with receptacle 106. In at least one embodiment, channel manifold 116 is attached to microfluidic cartridge subassembly 108 by fasteners, welding, or adhesion. In at least one embodiment, slotted cap 120 (described below) protrudes above upper panel 122 of microfluidic cartridge subassembly 108. Slotted cap 120 is coupled to a rotatable shaft of a rotatable valve (described below) extending from upper panel 122 to microfluidic manifold 116.

FIG. 1B illustrates a 3D isometric view of microfluidic subassembly 104, in accordance with at least one embodiment. In at least one embodiment, receptacle 106 is a hollow structure comprising cavity 124 surrounded by sidewall 110. In at least one embodiment, cavity 124 occupies the majority of the volume of receptacle 106. In at least one embodiment, threads 126 are disposed near rim 128 of receptacle 106. Threads 126 may be configured to engage mating threads (not shown) at the base of outer container 112 (FIG. 1A). In at least one embodiment, the threaded rim comprises female threads disposed on an interior surface of the sidewall. In at least one embodiment, the threads are male threads disposed on an exterior surface of the sidewall.

In other embodiments, threads may be replaced by other securing mechanisms, such as a bayonet-style coupling. In at least one embodiment, receptacle 106 comprises floor 130 at base of cavity 124.

In at least one embodiment, microfluidic cartridge subassembly 108 extends laterally, cantilevered from sidewall 110 of receptacle 106. Microfluidic cartridge subassembly 108 comprises upper enclosure panel 122 and channel manifold 116 as a lower panel. In at least one embodiment a gasket (not shown) may be attached over channel manifold 116 to seal open channels and chambers on channel manifold 116. In at least one embodiment, upper panel 122 is separated from channel manifold 116 by sidewalls 132A-132C. Sidewalls 132A-132C comprise frontal sidewall 132A and lateral sidewalls 132B and 132C. In at least one embodiment, sidewalls 132A-132C have a height h. Within sidewalls 132A-132C, microfluidic cartridge subassembly 108 may be mostly hollow, accommodating fasteners and a rotatable shaft of a rotatable selection valve coupled to channel manifold 116, described below.

In at least one embodiment, slotted cap 120 extends above upper panel 122. In at least one embodiment, slotted cap 120 is configured to engage a motor shaft (described below) to turn a rotatable selection valve configured to transfer fluids between compliant insert 114 and analysis chambers 118. The rotatable selection valve is described below. In the illustrated embodiment, analysis chambers 118A and 118B are shown extending below channel manifold 116.

FIG. 1C illustrates a rotated 3D view of microfluidic subassembly 104, showing lower portions of receptacle 106 and microfluidic cartridge subassembly 108, in accordance with at least one embodiment. In the illustrated embodiment, channel manifold 116 comprises analysis chambers 118A, 118B, and 118C. While three analysis chambers are shown, any suitable number of analysis chambers may be employed. In at least one embodiment, analysis chambers 118A-C are interconnected via channels 136, 138, and 140 that are routed to rotatable selection valve 134. As will be described below, rotatable selection valve 134 comprises an opening that communicates with a cavity within a rotatable shaft coupled to rotatable selection valve 134 (see FIG. 1F). A plunger is disposed within the cavity, enabling fluid transfer by pipetting action.

Channel 142 is routed to receptacle 106 at a terminus within indentation 144, where it communicates with cavity 124. In at least one embodiment, channel 142 terminates at an opening into cavity 124, where during operation fluid container 112 is positioned. A sample of growth medium, for example, contained within fluid container 112, may be withdrawn into channel 142 by pipetting action through rotatable selection valve 134. Further pipetting action may draw fluid to terminal 146 of channel 142, where an aliquot may be drawn through an opening (not shown) on rotatable selection valve 134, into the cavity within the rotatable shaft coupled to rotatable selection valve 134. Rotatable valve 134 may be rotated to align the opening to termini of channels 136, 138, or 140, enabling transfer of the aliquot to any of analysis chambers 118.

FIG. 1D illustrates an exploded view of microfluidic cartridge subassembly 104, in accordance with at least one embodiment. In at least one embodiment, microfluidic cartridge subassembly 108 comprises upper panel 122, lower panel 117, opposing upper panel 122, and sidewalls 132A, 132B, and 132C surrounding platform cavity 148. In at least one embodiment, rotatable shaft 150, coupled to rotatable selection valve 134, extends through bushing 152 to slotted cap 120 through opening 154 in upper panel 122. Slots 156 in upper portion of rotatable shaft 150 may be configured to engage slotted cap 120. In at least one embodiment, slotted cap 120 is seated over opening 154. In at least one embodiment, bushing 152 extends through platform cavity 148 between lower panel 117 and upper panel 122. In at least one embodiment, bushing 152 is integral with lower panel 117.

In at least one embodiment, microfluidic subassembly 104 includes gasket 158 that may be disposed between channel manifold 116 and lower panel 117. Both channel manifold 116 and gasket 158 may comprise through-holes 160 to enable passage of fasteners 162 through platform cavity 148. In at least one embodiment, fasteners 162 may pass through standoffs 164 extending between lower panel 117 and upper panel 122. Both gasket 158 and channel manifold 116 comprise tab 166 and tab 167, respectively, that fit into indentation 144 at the base of receptacle 106. In at least one embodiment, tab 166 of gasket 158 comprises an opening enabling channel 142 to communicate with cavity 124 of receptacle 106.

In at least one embodiment, the platform subassembly may be assembled by use of fasteners 162, as shown in the illustrated embodiment. In at least one embodiment, channel manifold 116 and upper panel 122 may be assembled to sidewalls 132A-C and lower panel 117 by an adhesive or by ultrasonic welding, for example, obviating the use of fasteners 162. In at least one embodiment, microfluidic cartridge subassembly 108 may comprise a molded piece comprising lower panel 117 and sidewalls 132, both integral with receptacle 106. Rotatable shaft 150 coupled to rotatable selection valve 134 may be inserted into bushing 152, also molded together with lower panel 117 and sidewalls 132, followed by attachment of gasket 158 and channel manifold 116. Upper panel 122 may be attached to sidewalls 132 to complete assembly.

The inset in FIG. 1D shows an overhead plan view of microfluidic cartridge subassembly 104, with upper panel 122 removed and showing terminal 168 of channel 142 communicating with cavity 124 of receptacle 106 through indentation 144. In at least one embodiment, terminal 168 may extend to tab 166 of channel manifold 116, where tab 166 conforms to indentation 144.

Also shown in the inset is slotted cap 120, where the plan view more clearly shows slot 170 across slotted cap 120. In at least one embodiment, slot 170 has a taper along its length. In at least one embodiment, analysis chamber 118A may have an opening 172 extending through upper panel 122.

FIG. 1E illustrates a perspective view of rotatable selector valve assembly 174 engaged with slotted cap 120, in accordance with at least one embodiment. In at least one embodiment, rotatable selector valve assembly 174 comprises rotatable selector valve 134 coupled to rotatable shaft 150. In at least one embodiment, opening 176 extends into an interior region of rotatable selector valve 134. In at least one embodiment, opening 176 is disposed in peripheral zone of rotatable selector valve 134. Slot 156 is shown engaged with slotted cap 120, which comprises tooth 178 to engage into slot 156, in accordance with at least one embodiment.

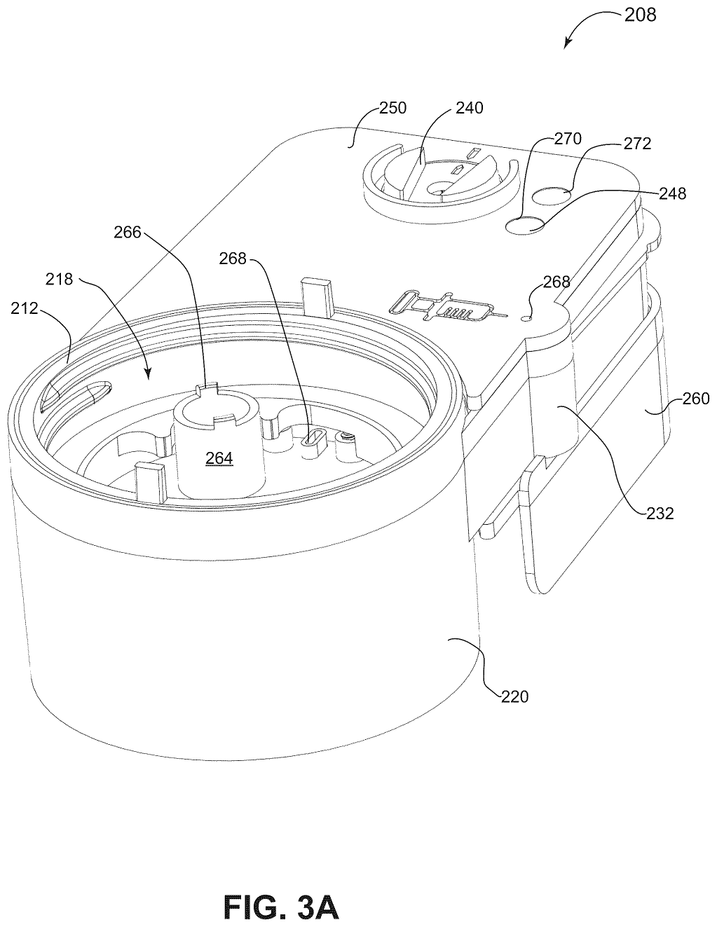

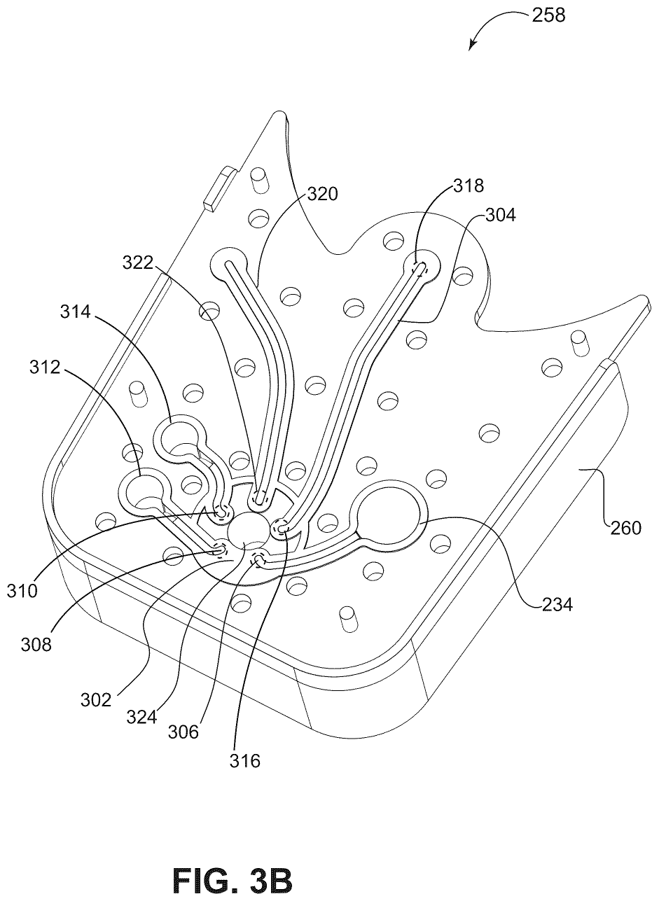

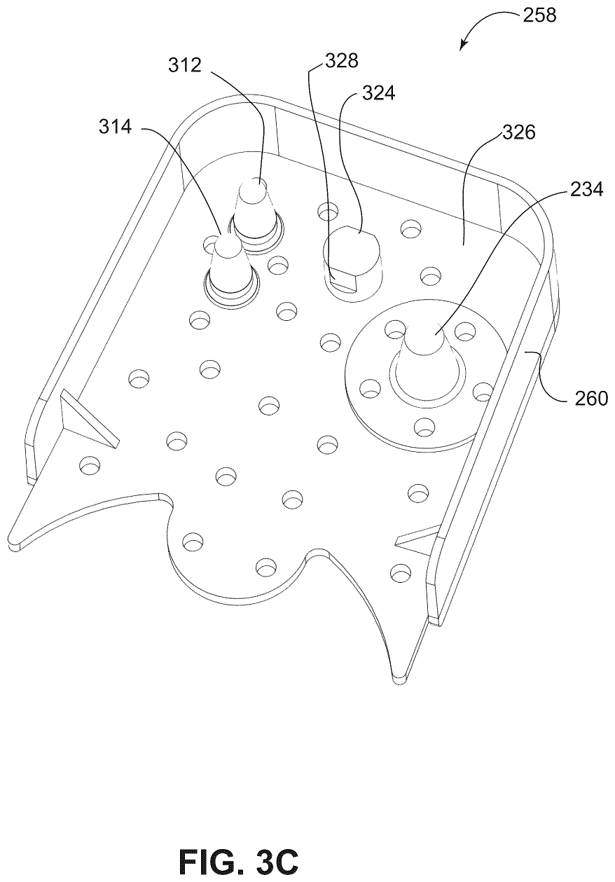

FIG. 1F illustrates a cross-sectional view of rotatable selector valve assembly 174, in accordance with at least one embodiment. In at least one embodiment, cavity 180 is disposed within rotatable shaft 150 and communicates with opening 176 through channel 182. In at least one embodiment, an opening in slotted cap 120 (not shown) enables passage of a plunger shaft into cavity 180. A compliant plunger tip (not shown) is confined within cavity 180 and is engageable with the plunger shaft. When engaged, the plunger tip is movable within cavity 180. The motion of plunger tip enables depressurization and pressurization of cavity 180, enabling transfer of aliquots of fluid by pipetting action, causing fluid to enter and exit cavity 180 through channel 182 and opening 176. Rotatable selector valve 134 may be abutted against a channel manifold (e.g., channel manifold 258, see FIG. 3B).

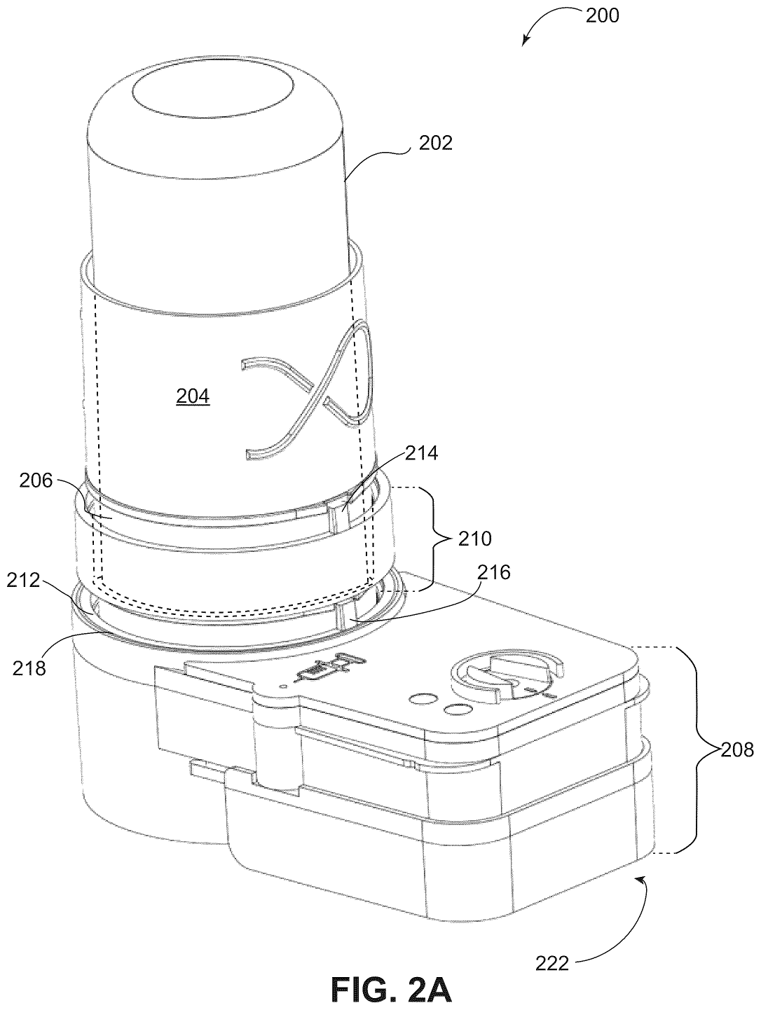

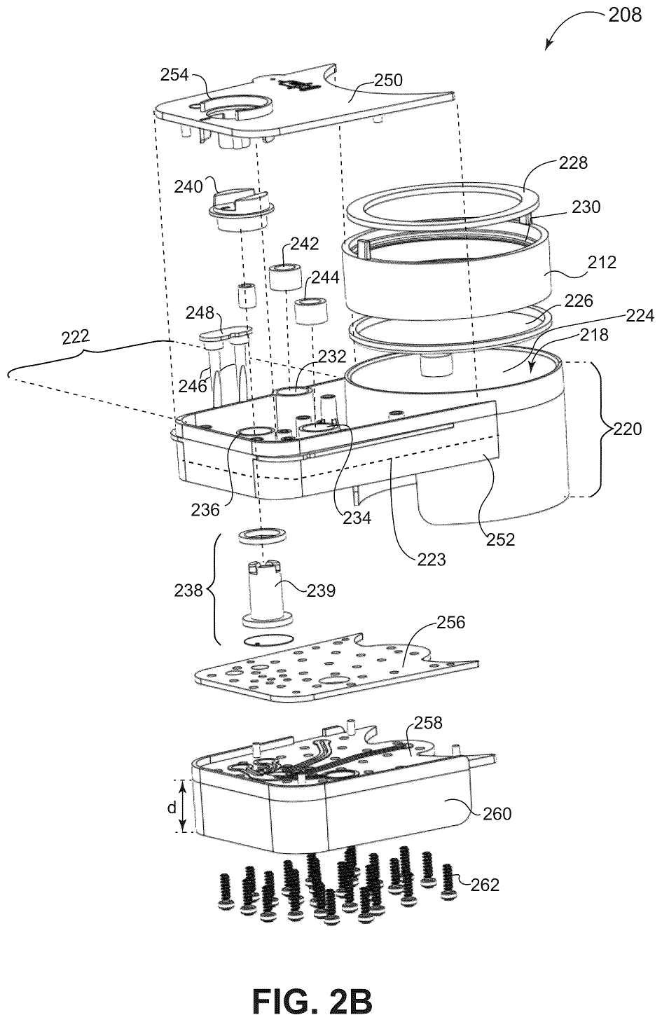

FIG. 2A illustrates a 3D view of self-contained microbiological analysis module (SCMAM) assembly 200, in accordance with at least one embodiment. Here, SCMAM assembly 200 comprises culture enrichment chamber 202 as part of a culture enrichment subassembly and a separate microfluidic cartridge subassembly (e.g., cartridge) 208. In at least one embodiment, culture enrichment chamber 202 is inserted through attachment collar 204 and accompanying socket 206 (e.g., socket 206 may be integral with attachment collar 204). Culture enrichment chamber 202 is shown to be inverted as its working configuration when assembled to microfluidic cartridge subassembly 208. In this configuration, the bottom portion of culture enrichment chamber 202 extends above attachment collar 204 and the top portion of culture enrichment chamber 202 extends below attachment collar 204. It is understood that culture enrichment chamber 202 is a separate component of the enrichment subassembly. Culture enrichment chamber 202 may be provided to the user as a sac that is partially filled with a liquid culture fluid and inoculated by a microbiological sample. In at least one embodiment, culture enrichment chamber 202 comprises a compliant plastic material, such as polyethylene, such that the wall is expandible and collapsible. Once inoculated, the other components of the enrichment subassembly, described below, are assembled together and the completed enrichment subassembly attached to cartridge subassembly 208 to complete SCMAM assembly 200.

Referring to the enrichment subassembly, in at least one embodiment, socket 206 is configured to fasten to base 210. The hidden lines in FIG. 2A indicate the upper portion of culture enrichment chamber 202 that may be hidden from view in a completed SCMAM assembly 200. Culture enrichment chamber 202 extends through attachment collar 204 and socket 206, terminating at its open mouth, which is ringed by a rim or lip. The rim may be pressed against an internal seal within base 210 to seal culture enrichment chamber 202 and retain liquid contents it may hold. For example, the rim of culture enrichment chamber 202 may seat over a shelf or seal within attachment base 210. As is described below, culture enrichment chamber 202 comprises a compliant material, such as high-density polyethylene sheet film, for example, enabling expansion and collapse of its volume. Attachment collar 204 is a rigid structure, comprising a ridged thermoplastic or thermoset plastic material, for example.

In at least one embodiment, base 210 may fasten into cartridge adapter 212. A membrane or other seal may be included to hermetically close the bottom of base 210 so that contents of culture enrichment chamber are contained. In at least one embodiment, attachment collar 204 locks to base 210 by upper locking tab 214. In at least one embodiment, base 210 may lock to cartridge adapter 212 by lower locking tab 216. Upper locking tab 214 and lower locking tab 216 may be positioned to engage with detents on their respective mating parts such that a sufficient pressure is exerted on the rim of culture enrichment chamber 202 by rotation of socket 206 and by rotation of base 210 on any seals residing within cartridge adapter 212. While not shown in detail, the detents may have ramps on either side of the detent that allow bidirectional twisting of attachment collar 204 or socket 206 to align upper and lower locking tabs 214 and 216, respectively, with detents. In this manner, desired torques may be obtained by rotational adjustment of attachment collar 204 and socket 206.

Cartridge adapter 212 may comprise a ring body that fastens or is press-fit into aperture 218 of microfluidic cartridge subassembly 208. Mechanical stability and a hermetic seal between the enrichment subassembly and microfluidic cartridge subassembly 208 may be formed in this manner. Liquid contents of enrichment chamber 202 may be contacted to internal surfaces of microfluidic cartridge subassembly 208 by cutting open or puncturing a scaling membrane (not shown but described above) internally by a blade or other sharp protrusion within the cavity of aperture 218 into which cartridge adapter 212 is pressed to attach the enrichment subassembly to microfluidic cartridge subassembly 208.

FIG. 2B shows an exploded 3D view of microfluidic cartridge subassembly 208, in accordance with at least one embodiment. Culture enrichment chamber receptacle (receptacle) 220 is a cylindrical structure supporting microfluidic stage 222 that is cantilevered from one side of receptacle 220. Receptacle 220 comprises aperture 218, into which cartridge adapter 212 may be press-fit to attach the enrichment subassembly to microfluidic cartridge subassembly 208, as noted above. While cartridge adapter 212 is shown in the illustrative embodiment to be configured to press fit into aperture 218 aperture 218, whereby cartridge adapter wall is contacted to interior wall 224 of aperture 218, in some embodiments, cartridge adapter 212 may also be configured with external threads to fasten to aperture 218.

At the base of cartridge adapter 212, a membrane 226 may be placed or inserted prior to insertion of cartridge adapter 212 to aperture 218. Membrane 226 may serve to contain fluid within enrichment subassembly (notably the enrichment fluid contained within culture enrichment chamber 202). A seal 228 (or gasket) may be inserted on the upper portion of cartridge adapter 212, to seat on an interior shelf 230, for example, providing a seal for the lip of culture enrichment chamber 202.

Microfluidic stage 222 comprises midplane panel 223 (indicated by the hidden line shown extending through sidewall panels of microfluidic stage 222), where upon prime chamber 232 and reagent chamber 234 are located and in some embodiments are integral therewith. The purpose of these chambers is described below. Microfluidic stage 222 further comprises rotational valve bushing 236, also integral with midplane panel 223, allowing assembly of rotatable selector valve shaft 239 to microfluidic stage 222. Rotatable selector valve shaft 239 is configured for attaching to valve handle 240, which provides an engagement for mechanical manipulation shaft (e.g., plunger shaft 546) of rotatable valve shaft 239. Plugs 242 and 244 are configured to insert into prime chamber 232 and lysing chamber 234, respectively.

In at least one embodiment, a pair of light guides 246 are mounted through the midplane panel 223 of microfluidic stage 222. Light guides 246 have substantially a rod shape, and may be integral with optical window 248. In at least one embodiment, the light guide assembly comprises light guides 246 attached to optical window 248 as a unitary molded part formed from a polymer that exhibits sufficient transparency to visible and/or ultraviolet light wavelengths of interest for analytical purposes. As will be described below, light guides 246 may also serve to guide liquid reagents into reaction vessels (e.g., analysis chambers 312 and 314, FIG. 3B) during fluid transfer operations.

In some embodiments, microfluidic stage 222 comprises upper panel 250, located above midplane panel 223. Upper panel 250 may comprise a rigid plastic material similar to that of receptacle 220 and stage sidewall 252. Upper panel 250 provides a cover for the internal components of microfluidic stage 222 located on midplane panel 223. An opening 254 in upper panel 250 is provided for valve handle 240 to protrude above upper panel 250.

Below the floor of microfluidic stage 222, a channel manifold layer is placed proximally to prime and reagent chambers 232 and 234, respectively, as well as rotational valve bushing 236. The fluid manifold layer comprises gasket 256 over and channel manifold 258 provides a platform for fluid transfer within SCMAM assembly 200. Channel manifold 258 is located below midplane panel 223, and comprises microchannels and reaction/reagent vessels, as described below. Channel manifold 258 may be supported by skirt 260, which extends below channel manifold 258 a distance d to provide a stable stand for SCMAM assembly 200 when assembled. Skirt 260 may open on the bottom to allow access to reaction and reagent vessels. In the illustrative embodiment, while fasteners 262 are shown as the means to assemble all the parts described above, other means to affix parts together may be employed, such as ultrasonic welding.

FIG. 3A shows a 3D isometric view of microfluidic cartridge subassembly 208, in accordance with at least one embodiment. The isometric view of FIG. 3A shows some inner components of aperture 218. Culture termination chamber 264 is positioned such that it is offset from the center of aperture 218. Tabs 266 extend above the rim of chamber 264. In at least one embodiment, tabs 266 have a knife or cutting edge on an upper edge that permits puncture and cutting open membrane 226 (see FIG. 2B) to enable liquid contents of culture enrichment chamber 202 to spill into aperture 218. Culture termination chamber 264 may contain a plug (not shown) to prevent premature contact of enrichment fluid with an antimicrobial substance in the form of a tablet, powder, or liquid contained within culture termination chamber 264. Opening 268 extends through the floor of aperture 218 to align with channel manifold 258 below the level of the aperture floor for enabling intake of culture enrichment fluid samples.

In at least one embodiment, upper panel 250 comprises opening 268 over prime chamber 232, which may protrude laterally from microfluidic stage 222, as shown. This feature will be described below. Opening 268 may be provided for insertion of a syringe needle to procure enrichment fluid samples that are retained within prime chamber 232 for external analysis. For example, manual sampling of the culture enrichment fluid may be performed by taking aliquots with a syringe, for example, to identify species of microbe(s) that are in the sample by nucleic acid sequencing, and for other external assays.

Upper panel 250 also may comprise openings 270 and 272 for access to optical window 248. Optical window 248 forms a cap over analysis chambers (e.g., analysis chambers 312 and 314) in channel manifold 258. Openings 270 and 272 are aligned to tops of the analysis chambers. In an example, for fluorescent assays, light from fluorescence analyses may be sampled through openings 270 and 272 by photodetectors positioned above openings 270 and 272, where fluorescence may be engendered by appropriately positioned LEDs shining into reaction vessels, as will be described below. In another example, light may be shined through openings 270 and 272 and directed into reaction vessels by light guides 246 to determine turbidity of reaction fluid or state of dissolution of solids by appropriately placed photodetectors. Light, either introduced through openings 270 and 272 or sampled from opening 270 and 272, is transferred to or from reaction vessels through rod-shaped light guides 246. In some embodiments, an optical filter to allow passage of a narrow band of visible or ultraviolet wavelengths may be inserted over optical window, or incorporated therein, to allow passage of only a predetermined color of light to avoid stray light interference of analyte detection.

FIG. 3B illustrates a 3D view of channel manifold 258, in accordance with at least one embodiment. Skirt 260 is attached as shown. A stator base 302 of rotatable selector valve assembly 238 is integral with channel manifold 258 and serves as the channel nexus. Through stator base 302, the channels comprising the manifold, such as sample channel 304 on channel manifold 258, communicate with fluid passages of the rotatable valve (e.g., rotatable selector valve 134, see FIG. 1E) portion of rotatable selector valve assembly 238. Rotatable selector valve assembly 238 is configured substantially as rotatable selector valve assembly 174, described above. For example, an inlet (e.g., opening 176, see FIG. 1E) on rotatable selector valve assembly 238 may be rotated over channel termini, such as channel terminal 306, to select routing of fluids. For example, with the inlet (e.g., opening 176) positioned over channel terminal 306, bacterial cellular or viral coat lysis fluid may be withdrawn from lysis chamber 234 using the pump action of rotatable selector valve assembly 238 described above. Aliquots of lysis fluid may be transferred to channel termini 308 or 310, which can then be pumped to analysis chambers 312 or 314, respectively. Similarly, enrichment fluid samples may be obtained by rotation of rotatable selector valve shaft 239 to align with channel terminal 316 of sample channel 304. Channel terminal 318 at the opposite end of sample channel 304 is in fluidic communication with aperture 218, shown in FIGS. 2B and 3A, in particular through opening 268 (see FIG. 3A) on the floor of aperture 218. The enrichment fluid samples may be transferred as aliquots of predetermined volumes to analysis chambers 312 or 314. Enrichment fluid samples may also be transferred to prime chamber 232 via channel 320 if external assaying is desired. Rotatable selection valve shaft 239 may be positioned over channel terminal 322 from channel terminal 316 and pumped through channel 320 to prime chamber 232.

In at least one embodiment, stator base 302 comprises overflow chamber 324. Overflow chamber 324 may provide a sump or well into which fluids that leak from rotatable selector valve shaft 239 may drain and be retained. In this way, cross contamination can be avoided, and if fluids do accumulate in overflow chamber, the assay may be terminated.

FIG. 3C illustrates a reverse side of channel manifold 258, in accordance with at least one embodiment. Overflow chamber 324 and analysis chambers 312 and 314 are shown protruding from the lower side 326 of channel manifold 258. In some embodiments, flats 328 on overflow chamber 324 are provided as optical windows. Any leaking fluids that enter overflow chamber 324 may be monitored optically by shining light from an LED through flats 328. In the event fluids accumulate in overflow chamber 324, light from an LED that shines through flats 328 would be deviated or scattered by refraction and any turbidity in the optical path due to the accumulated fluid. Skirt 260 provides a flat base upon which the entire SCMAM assembly 200 may be set. Channel manifold 258 is not covered from below to allow access to chambers for insertion into wells in an analysis station (e.g., analysis station instrument 500, described below) for optical assays, such as fluorescent assays and self-monitoring of chambers for undissolved reagent tablets and other solids in analysis chambers 312 and 314, as well as fluids entering overflow chamber 324.

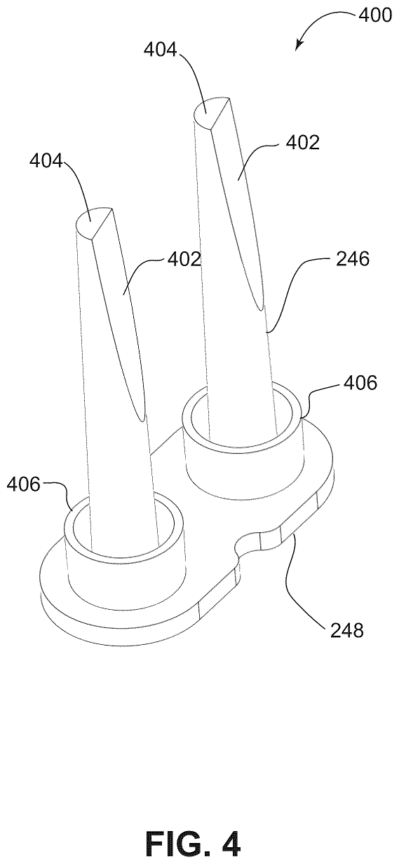

FIG. 4 illustrates light guide assembly 400, comprising light guides 246 integral with optical window 248, in accordance with at least one embodiment. In the illustrative embodiment, light guide assembly 400 is inverted to show details. In at least one embodiment, light guides 246 have a tapered flat 402 extending to bottoms 404 of light guides 246. Light guides 246 are configured to insert into analysis chamber 312 and 314 to bring light into or out of analysis chambers 312 and 314. Tapered flats 402 may provide guides for fluid entry into analysis chambers 312 and 314 during fluid transfer, causing fluids to flow down light guides 246 and accumulate in the lower portion of analysis chambers 312 and 314. In some embodiments, rings 406 extend from optical window 248, surrounding the upper portions of light guides 246. During assembly, rings 406 are pressed form a hermetic seal with analysis chambers 312 and 314.

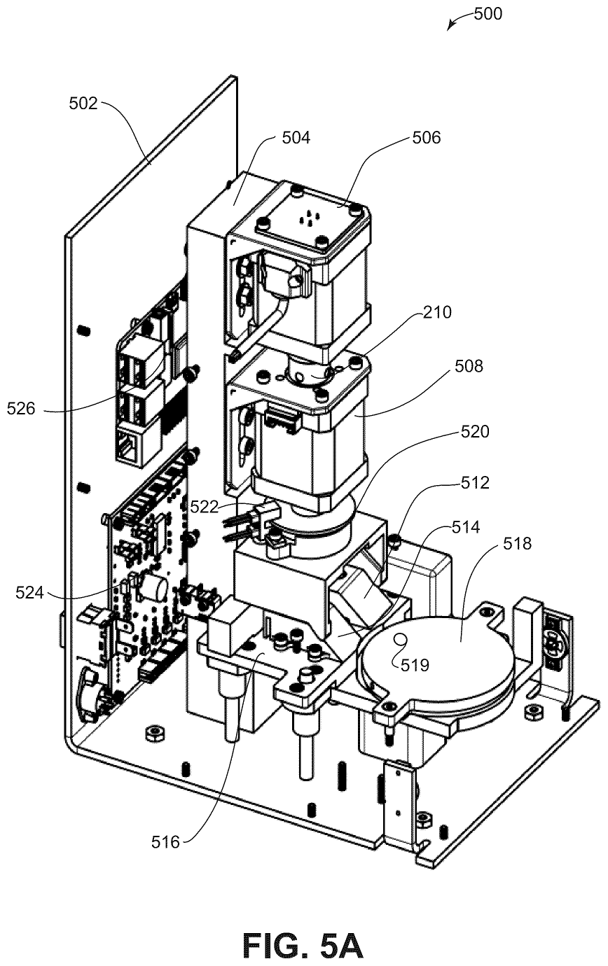

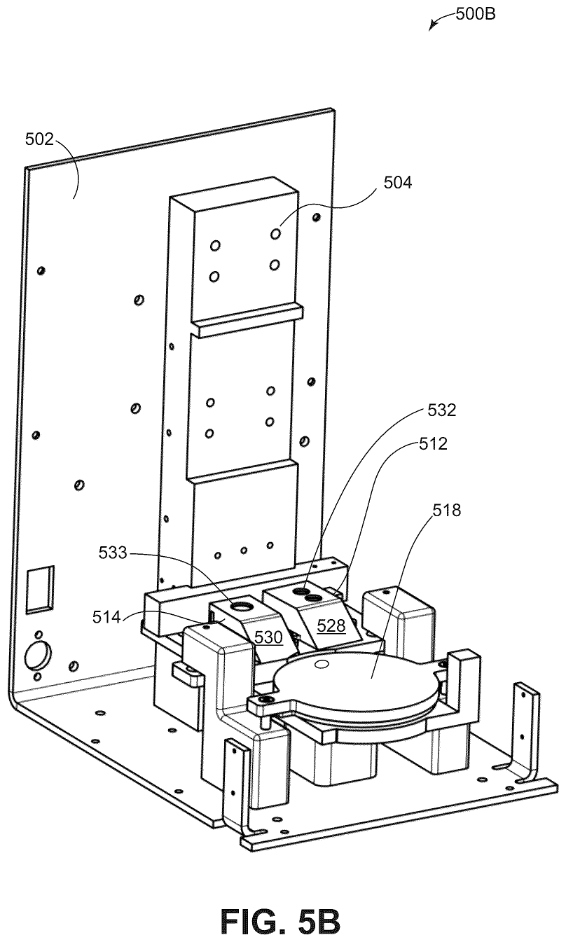

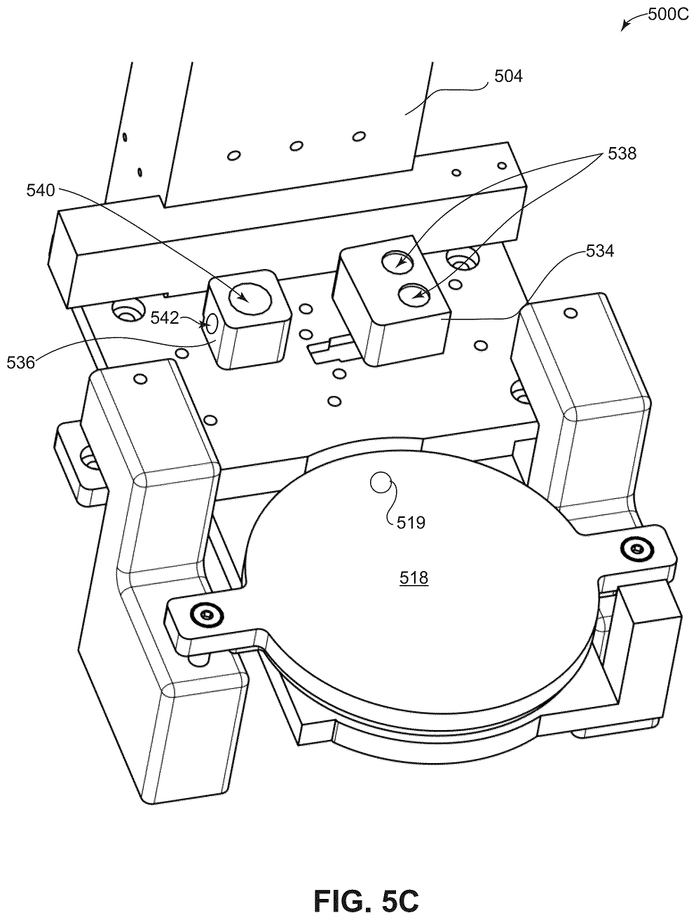

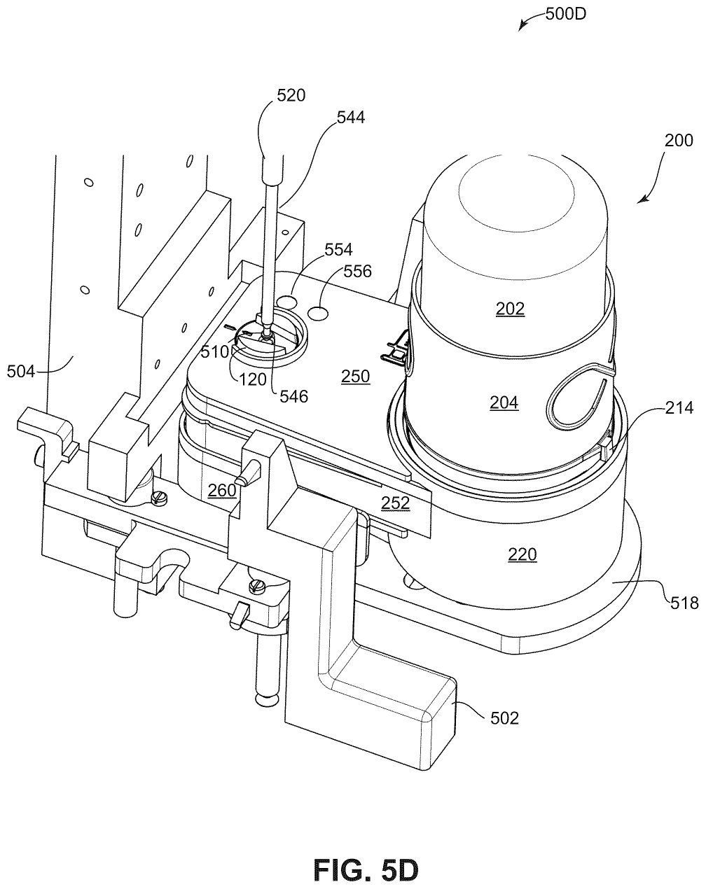

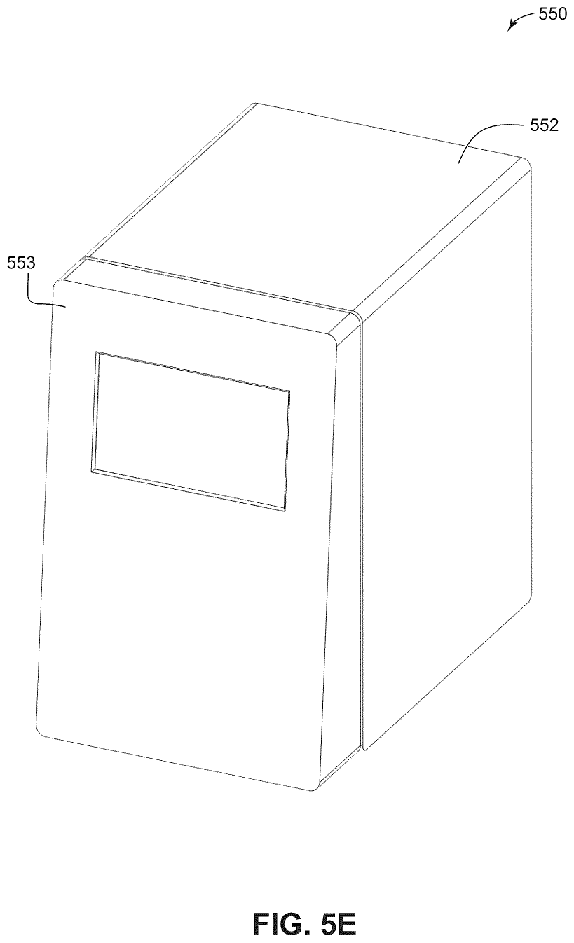

FIG. 5A illustrates a perspective view of analysis station instrument 500, in accordance with at least one embodiment. In at least one embodiment, analysis station instrument 500 comprises chassis 502, mounting column 504 affixed to chassis 502. In at least one embodiment, plunger shaft driver motor (not shown) and rotatable motor 508 are affixed to mounting column 504. In at least one embodiment, shaft 510 from plunger shaft driver motor 506 extends through rotatable motor 508 to extend below. In at least one embodiment, analysis station instrument 500 comprises a docking portion for engaging SCMAM assembly 200. Within the docking portion, ramps 512 and 514 are supported on stage 516.

In at least one embodiment, analysis station instrument 500 further comprises stage 518 onto which receptacle 106 or 220 (e.g., sec FIGS. 1A, 2B or 3A) may seat when SCMAM assembly 100 or 200 is engaged with analysis station instrument 500. In at least one embodiment, an access hole 519 extends through a portion of stage 518. Access hole 519 may be located at a position that is directly under culture terminal chamber 264 within receptacle 220 (e.g., see FIG. 3A) for access by a stylus or other instrument to push an antimicrobial tablet into the cavity of aperture 218. In at least one embodiment, analysis station instrument 500 further comprises encoder 520. In at least one embodiment, encoder 520 is attached to a shaft (not shown) of rotatable motor 508. In at least one embodiment, encoder 520 is configured to provide a measure of rotatable motion of rotatable selection valve 134. For example, marks on encoder 520 are recorded by optical sensor 522, enabling precision control of rotatable motion of rotatable selection valve 134 for fluid transfer on channel manifold 116 during operation.

In at least one embodiment, analysis station instrument 500 further comprises circuit board 524 and circuit board 526 affixed to chassis 502. While two circuit boards are shown in the exemplary embodiment, any suitable number of circuit boards may be employed. In at least one embodiment, circuit boards 524 and 526 may be configured to provide analog control, power and digital logic for detectors, sensors, and motor drives. In at least one embodiment, circuit boards 524 and 526 may comprise on-board microprocessors, or portals via wifi, ethernet, Bluetooth, USB, and I2C serial architectures, for example, for connections to external computers for control and data acquisition. In at least one embodiment, circuit board 526 may be a single board computer (e.g., Raspberry Pi, Beagleboard, etc.), enabling fully self- contained operation independent of an external computer.

In at least one embodiment, instrumentation control and data analysis may be combined within a processor on board the single board computer or external computer. In at least one embodiment, the processor may be a Digital Signal Processor (DSP), an Application Specific Integrated Circuit (ASIC), a general-purpose Central Processing Unit (CPU), or a low power logic implementing a simple finite state machine to perform various processes described herein. In at least one embodiment, a tangible machine-readable storage medium (not shown) is electronically coupled to the processor. Examples of a tangible machine-readable storage medium include, but are not limited to, recordable and non-recordable type media, such as volatile and non-volatile memory devices, read only memory (ROM), random-access-memory (RAM), flash memory devices, floppy and other removable disks, magnetic storage media, optical storage media (e.g., Compact Disk Read-Only Memory (CD ROMS), Digital Versatile Disks (DVDs), etc.), among others. The machine-readable medium has machine-readable instructions, that when executed, cause the processor to perform the method as discussed with reference to various embodiments. In at least one embodiment, machine-readable storage medium may include instructions (also referred to as the program software code/instructions), such as instructions for the detection of a microorganism in a sample.

In at least one embodiment, the processor may be operable to execute software code for reading pressure sensors, temperature sensors, light detector, and for issuing digital commands to light source, light detector, and other devices described herein. In at least one embodiment, the processor may be combined on a single printed circuit board (PCB) with a light source and light detector (or integrated optical system). In at least one embodiment, the processor may further command plunger driver motor 506 and rotatable motor 508, for example.

In at least one embodiment, analysis station instrument 500 may further comprise miniaturized analytical instrumentation. In at least one embodiment analysis station instrument 500 may comprise a spectrometric analysis unit (e.g., comprising a light source and light detector). In at least one embodiment, the light source and light detector may be separate stand-alone devices. In at least one embodiment, the light source may be a solid-state laser light source. In at least one embodiment, the light source may be a solid-state light emitting diode (LED) visible light or ultraviolet light laser. In at least one embodiment, the light source may be a stand-alone laser. In at least one embodiment, the light detector may be an integrated optical spectrometer. In at least one embodiment, the light detector may be a photodiode, for example, if wavelength resolution of the received light is not desired.

FIG. 5B illustrates a perspective view 500B of a portion of analysis station instrument 500, in accordance with at least one embodiment. In at least one embodiment, view 500B shows a stripped down view of analysis station instrument 500, exposing ramps 512 and 514. In at least one embodiment, ramps 512 and 514 comprise ramped sidewalls 528 and 530, respectively. In at least one embodiment, ramped sidewalls may have a slope between 30 and 50 degrees, configured to guide analysis chambers 118 into openings 532 and 533 during docking of SCMAM assembly 100.

FIG. 5C illustrates a perspective view 500C, showing an enlarged view of analysis station instrument 500, in accordance with at least one embodiment. In view 500C, ramps 512 and 514 are removed to show heating blocks 534 and 536. In at least one embodiment, heating blocks 534 and 536 are disposed within hollow interiors of ramps 512 and 514, respectively, to engage analysis chambers 118 extending below channel manifold 116. In at least one embodiment, a vibrating plate (not shown) may be adjacent one or both heating blocks 534 and/or 536. A vibrating plate, for example a piezoelectrically driven vibrating plate, may be configured to aid dissolution of powders, for example, contained within analysis chambers 118. In at least one embodiment, heating block 534 comprises chambers 538 configured to fit a pair of analysis chambers (e.g., analysis chambers 118B and 118C) extending below channel manifold 116.

In at least one embodiment, heating block 536 comprises chamber 540. In at least one embodiment, chamber 540 is configured to accommodate a analysis chamber, such as analysis chamber 118A. In at least one embodiment, heating blocks 534 and 536 comprise view holes, such as view hole 542 on the side of heating block 536. In at least one embodiment, view holes similar to view hole 542 are disposed on a hidden side of heating block 534. In at least one embodiment, heating blocks 534 and 536 comprise view holes (not shown) at the bottom of chambers 538 and 540, respectively. In at least one embodiment, view holes function to pass light to excite fluorescence of analyte molecules within analysis chambers 118. For example, excitation light beam may enter chambers 538 and/or 540 through side view holes, such as view hole 542. Fluorescent light may be detected through view holes at the bottom of chambers 538 and 540, so that fluorescent light is orthogonal to excitation light avoiding interference by the latter since the excitation beam may not scatter in that direction.

FIG. 5D illustrates a perspective view 500D of analysis station instrument 500, in accordance with at least one embodiment. In at least one embodiment, SCMAM assembly 200 is docked and engaged within analysis station instrument 500. Rotatable motor 508 is removed to show sheath 544 extending downward from plunger drive motor 506. In at least one embodiment, plunger shaft 546 extends through sheath 544, configured to house plunger shaft 546. In at least one embodiment, plunger shaft 546 extends through valve handle 240. As described above, plunger shaft 546 is configured to engage a compliant plunger tip within cavity 180 of rotatable shaft 150 of rotatable selector valve assembly 174 (not shown). In at least one embodiment, plunger drive motor 508 is configured to move plunger shaft 546 vertically to cause displacement of the plunger tip for fluid transfer, as described above. LED light sources or photodetectors (not shown) may be positioned over apertures 554 and 556, which are openings to the optical window (e.g. optical window 248) and aligned to analysis chambers below through light guides 246.

FIG. 5E illustrates a perspective view of enclosure 550, in accordance with at least one embodiment. Enclosure 550 houses analysis station instrument 500, whereby chassis 502 and mounting column 504 are attached thereto. Enclosure comprises cabinet 552 and access door 553. In at least one embodiment, enclosure 550 provides a temperature-controlled cabinet for analysis station instrument 500. In at least one embodiment, enclosure 550 comprises a refrigeration unit to maintain refrigeration temperatures, such as 4° C.

In at least one embodiment, enclosure 550 comprises a cold finger or other refrigerated extension (not shown) to provide thermal contact to prime chamber 234 in order to maintain its contents at 4° C., for example.

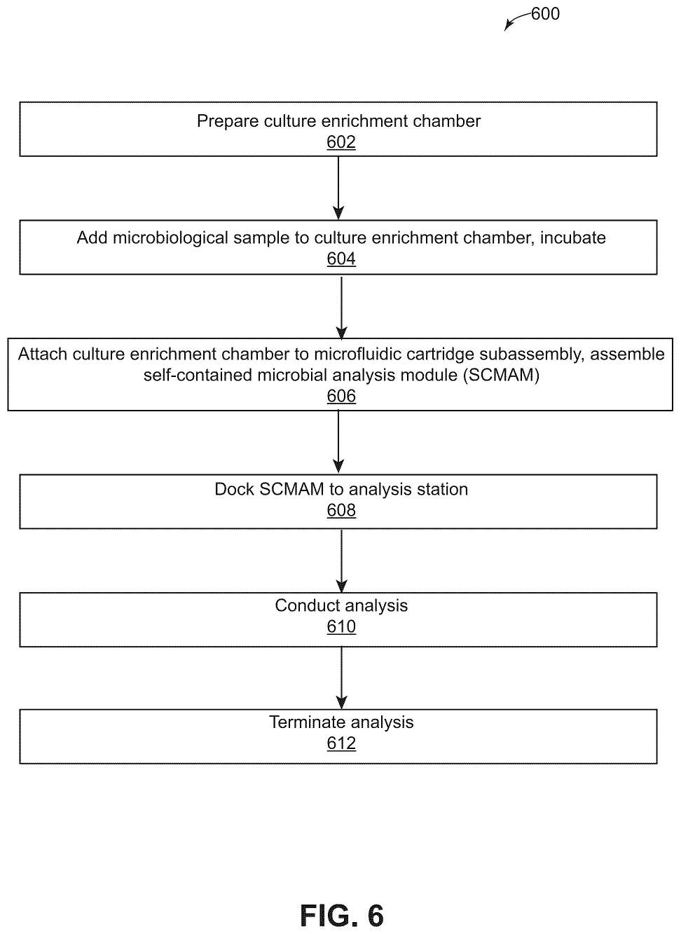

FIG. 6 illustrates flow chart 600 summarizing an exemplary method for operating analysis station instrument 500, in accordance with at least one embodiment. In at least one embodiment, the various blocks shown here can be implemented in hardware, software, or a combination thereof. In at least one embodiment, analysis station instrument 500 may enable automated fluid transfer from culture enrichment chamber 102 or 202 to any analysis chambers 118, 312, or 314. In at least one embodiment, analysis station instrument 500 may further comprise an incubation oven (not shown) to incubate media that may be contained within culture enrichment chamber 102 or 202. In at least one embodiment, analysis station instrument 500 may comprise a refrigeration unit for maintain refrigeration temperatures (e.g., 4° C.).

At operation 602, a compliant insert, such as compliant insert 114, may be inserted into outer container 112 to assemble culture enrichment chamber 102 subassembly. Alternatively, culture enrichment chamber 202 may be inserted into attachment collar 204. At this stage, the subassembly is inverted such that the contents may be retained and not spilled. (e.g., the mouth of culture enrichment chamber 102 or 202 is upright). The compliant insert 114 or culture enrichment chamber 202 may comprise a pre-sterilized sac prefilled with a liquid growth medium. In at least one embodiment, the culture enrichment chamber (e.g., compliant insert 114 or culture enrichment chamber 202) may be stored empty and filled with a liquid growth medium when deployed. In at least one embodiment, outer container 112 or attachment collar 204 is a sufficiently rigid structure to provide protection for the compliant chamber. In at least one embodiment, outer container 112 comprises a vent hole to enable pressure equalization for compliant insert 114.

Alternatively, in at least one embodiment, attachment collar 204 is fastened to socket 206. Socket 206 may be fastened to base 210. Base 210 may be fastened to cartridge adapter 212. For both attachment points, locking tabs (e.g., locking tabs 214 and 216) may be engaged to ensure adequate torque.

At operation 604, a microbiological sample is placed within compliant insert 114 or culture enrichment chamber 202. A microbiological sample may include a liquid or solid material harboring or suspected of harboring certain microorganisms. Microorganisms may be benign or pathogenic. In at least one embodiment, the microbiological sample may be inoculated or by insertion of a solid object. In at least one embodiment, an incubation period is imposed to allow a growth of microorganisms to a pre-determined density.

At operation 606, the fluid container subassembly is attached to microfluidic cartridge subassembly (e.g., microfluidic cartridge subassembly 104 or 208) to produce an analysis assembly, such as SCMAM assembly 100 or SCMAM assembly 200. In at least one embodiment, the culture enrichment chamber subassembly may be attached for example by engagement of mating threads on rim of outer container with rim of receptacle 106, or by insertion of cartridge adapter 212 into aperture 218 of SCMAM assembly 200. For the latter, cartridge adapter 212 may be press-fit into the cavity of aperture 218, whereby friction with wall 224 maintains integrality. Prior to analysis, the SCMAM assemblies 100 or 200 may be kept within an incubator or heated closet to incubate the culture for a predetermined period of time.

At operation 608, SCMAM assembly 100 or SCMAM assembly 200 may be docked with receiving structures within analysis station instrument 500 after an initial incubation period. In at least one embodiment, SCMAM assembly 100 is caused to slide into analysis station instrument 500, such that microfluidic cartridge subassembly 108 engages heating blocks 534 and 536 by contacting analysis chambers 118 to ramped sidewalls 528 and 530. Bottoms of analysis chambers 118 may slide up ramped sidewalls, then drop into chambers 538 and 540. In at least one embodiment, receptacle 106 is seated on stage 518. At this point, SCMAM assembly 100 is docked in analysis station instrument 500. Microfluidic cartridge subassembly 108 is positioned such that motor shafts, such as plunger shaft 546 is aligned to a center opening in slotted cap 120. In some embodiments, ramped sidewalls 528 are omitted, and stage 518 as well as heater blocks 534 and 536 are raised by a motor when SCMAM assembly 200 is seated.

Alternatively, in at least one embodiment, ramped sidewalls 528 and 530 may be omitted. SCMAM assembly 200 may be manually seated on stage 518 without sliding action, whereby analysis chambers 312 and 314 and reagent chamber 234 are aligned with and inserted into chambers 538 and 540, respectively and simultaneously, as aperture 218 is seated on stage 518. For example, SCMAM 200 is seated upon stage 518 and analysis chambers are aligned to chambers 538 and 540 in heater blocks 534 and 536, respectively. A motor may lift the subassembly comprising stage 518 and heater blocks 534 and 536 to a predetermined position.

At operation 610, an analysis may be initiated. Exemplary analyses may include cell lysis in one analysis chamber, such as analysis chamber 118A (or analysis chamber 312, SCMAM assembly 200). In some embodiments, a mixing action may be assisted by actuating by a vibrating plate on stage 518, for example. In some embodiments, analysis chambers may be pre-charged with a solid tablet or powder containing lysis enzyme, for example. In one example, liquid reagent from reagent chamber 234, for example, may be transferred into the analysis chamber (e.g., analysis chamber 312) from reagent chamber (e.g., reagent chamber 234). The liquid reagent may comprise a pH buffer and other ingredients necessary for the reaction to proceed. In an alternate example, analysis chamber may be initially empty, and the reagent held within reagent chamber may have pre-dissolved lysis enzyme and pH buffer.

Referring to SCMAM assembly 200, for example, transfer of fluid from the reagent vessel may be accomplished by rotation of rotatable selector valve assembly 174 (or rotatable selector valve assembly 238) to align opening 176 with a channel terminal (e.g., channel terminal 306) for the channel leading to reagent chamber 234. A plunger tip within cavity 180 of rotatable selector valve (e.g., rotatable selector valve assembly 238) may be engaged and raised by the plunger shaft (e.g., plunger shaft 546) of analysis station instrument 500. This action can pull an aliquot from reagent chamber 234 into cavity 180 of rotatable selector valve shaft 239 (or rotatable shaft 150). The opening 176 of the rotatable valve may be rotated to terminal 308 or 310 to transfer the aliquot of reagent fluid to analysis chamber 312 or 314 by lowering the plunger tip within cavity 180.

In at least one embodiment, as pre-charged lysis ingredients in the form of a powder or tablet are dissolving in a first analysis chamber, such as analysis chamber 312, the dissolution process may be monitored by shining light into the analysis chamber from the top. The light may be shined into optical window 248 and down light guides 246, for example, into the interior of analysis chamber 312. Light guides 246 may efficiently guide the light into the interior of analysis chamber 312 (or analysis chamber 314). As a secondary action, light guides 246 may mechanically restrain a tablet to the bottom portion of the analysis chamber, where otherwise it may float up to the top of the liquid by agitation or buoyancy effects. The light may be recorded by a photodetector positioned below or adjacent to the analysis chamber (e.g., analysis chamber 312 or 118B). The light wavelength for the purpose of monitoring the dissolution process may be a visible wavelength chosen so as not to excite fluorescence. By measuring passage of light through the analysis chamber as the solid material dissolves, completeness of dissolution may be determined by intensity of light impinging on the photodetector. For example, if the transmitted light intensity meets or exceeds a predetermined value, then dissolution may be deemed complete. Transmitted light intensity that falls below the predetermined value may provide a red flag indicating incomplete dissolution, and a (potentially) failed assay. In this situation, the assay may be aborted by the instrument (analysis station instrument 500).

Following the above example, upon a generation of a positive signal for dissolution, an aliquot of culture transfer of fluid from the receptacle (e.g., aperture 218) may be accomplished by rotation of rotatable selector valve assembly 174 (or rotatable selector valve assembly 238) to align opening 176 with a channel terminal (e.g., channel terminal 316) of sample channel 304. A plunger tip within cavity 180 of rotatable selector valve (e.g., rotatable selector valve assembly 238) may be engaged and raised by the plunger shaft (e.g., plunger shaft 546) of analysis station instrument 500. This action can pull an aliquot from aperture 218 into sample channel 304 and into cavity 180 of rotatable selector valve shaft 239 (or rotatable shaft 150). The opening 176 of the rotatable valve may be rotated to terminal 308 or 310 to transfer the aliquot to analysis chamber 312 or 314 by lowering the plunger tip within cavity 180. Here, the sample aliquot may be mixed by active mixing (e.g., vibration) or by diffusion with the lysis reagent. The mixing process may be heat assisted, by heating heater blocks 534 and/or 536.

In at least one embodiment, the analysis station may also be operable to control the temperature within the chambers. Heat may be added to activate the lysis step by switching on power to heating block 534, for example. In this manner, a sample preparation stage, such as bacterial lysis, may proceed at an optimal temperature within the analysis chamber as a sample preparation step, according to at least one embodiment. In at least one embodiment, the temperature may be ramped and cooled for specified durations (e.g., time periods), following a desired protocol. In at least one embodiment, the temperature can be controlled by resistive heating, heating by a Peltier element, or heating by circulating a temperature-controlled fluid that is heated in a heat exchanger within the base unit and routed to corresponding chambers containing the analysis chambers.

Analyte measurements may be based on optical or electrochemical detection means. For example, fluorescence of specific analytes may be a sensitive signal for detection of the presence and quantification of those analytes. In at least one embodiment, analysis station instrument 500 may comprise a spectrometer and/or an optical detection cell comprising a photodiode or phototransistor. Light sources for optical analysis of analytes may include lasing and non-lasing LEDs capable of producing high-intensity visible or UV light within narrow ranges of wavelengths. For example, blue or ultraviolet LEDs may be employed for fluorescence excitation. In at least one alternative embodiment, optical signals may comprise measured absorption of light due to the presence of a colored analyte that absorbs specific wavelengths of light. Sources for such light may be a colored or white light LED or LED laser source having a narrow bandwidth. In at least one embodiment, the light source may be within the automated analysis station, providing coherent or incoherent light that may be transmitted through a sample preparation analysis chamber containing a colored analyte, or a fluorescent analyte. In at least one embodiment, blue laser light may shine through the wall of the analysis chamber to excite the fluorophore portion of the fluorescent analyte. In at least one embodiment, fluorescent light emanating from the analyte may be measured by a spectrometer or photodiode.

In at least one embodiment, an example assay is a polymerase chain reaction (PCR) assay for identifying bacterial or viral pathogens. In at least one embodiment, lysis contents of the first analysis chamber (e.g., analysis chamber 312) may be heated for a specified or predetermined first duration. Lysis fluid within the first analysis chamber now contains free bacterial or viral DNA or RNA fragments after the cell or viral coat lysis step. In at least one embodiment, a sample of the lysed solution containing bacterial or viral nucleic acid (DNA or RNA) fragments and strands may then be transferred to a second analysis chamber, such as analysis chamber 314, by activation of the rotatable selector valve in an operational sequence described above, for, as an example, transfer of lysis solution from the reagent chamber to the first analysis chamber. The second analysis chamber may contain a PCR reagent medium, or a similar nucleic amplification system, such as isothermal PCR or loop-mediated isothermal amplification (LAMP).

In at least one embodiment, analysis station instrument 500 is programmed to transfer an aliquot of lysed cell solution to a second analysis chamber, such as analysis chamber 314. In at least one embodiment, once the sample is transferred from the first analysis chamber to the second analysis chamber containing the PCR medium (for example, by the automated sample transfer method described above), the temperature of the second sample preparation analysis chamber may be ramped to a programmed value for a specified or predetermined second duration. Within the second analysis chamber, a reaction may proceed by a second temperature regime applied to heater block 534. In at least one embodiment, during this time, the PCR reactions may occur within the second analysis chamber. In at least one embodiment, the PCR medium may contain a fluorescent marker molecule that binds with specific DNA strands or segments thereof that are unique to bacterial species of interest. The fluorescent marker fluoresces when excited by the light source emitting an excitation wavelength, such as blue or UV light.

In at least one embodiment, as noted above, fluorescent light may be detected by photodiode or phototransistor detectors located above analysis chambers 312 and 314. Fluorescence detection may be facilitated by light guides 246 within analysis chambers 312 and 314. Light guides 246 channel light from the solution to optical window 248 that is located above the PCR analysis chamber (e.g., analysis chamber 314). The light guide assembly (e.g., light guide assembly 400) may comprise a unitary structure, whereby light guides 246 are integral with optical window 248. Light guide assembly 400, for example, may also cap analysis chambers 312 and 314 for sealing purposes, whereby rings 406 may provide a hermetic sealing function.

In at least one embodiment, the second analysis chamber is within a chamber in the same or a second heating block. For example, fluorescent analytes may be generated by the reaction, which may be analyzed by shining an excitation light beam into the analysis chamber. In at least one embodiment, an excitation beam is introduced through a view hole extending through the analysis chamber, such as analysis chamber 312 or 314, seated within the heater block (e.g., heater block 534), in which the analysis chamber is seated. Fluorescent light is shone through a view hole, for example, on the bottom of the chamber. The fluorescence signal may be detected by measuring the orthogonally scattered light through optical window 248. In some embodiments, other optical signals may be employed, such as bioluminescence, turbidity and colorimetric signals.

At operation 612, after completion of the assay, an antimicrobial chemical may be internally released into the enrichment fluid to destroy any pathogens present in the fluid. This step may be performed prior to disposal of SCMAM assemblies 100 or 200. In at least one embodiment, this process may be accomplished by opening culture termination chamber 264 and releasing an antimicrobial powder, tablet or liquid containing sodium hypochlorite (bleach), or potassium iodide or iodate, for example. In at least one embodiment, a stylus may be pushed through culture termination access hole 519, which is located directly under culture termination chamber 264. In at least one embodiment, a tablet may be pushed out of culture termination chamber 264 by the action of the stylus. A chamber plug kept over the tablet during incubation and assay may be pushed out of culture termination chamber along with the tablet, where contact with the enrichment fluid activates the toxic ingredient.

Example 1 is a microbiological analysis assembly, comprising a culture enrichment chamber subassembly; and a microfluidic cartridge subassembly coupled to the culture enrichment chamber subassembly, wherein the culture enrichment chamber subassembly comprises a culture enrichment chamber, wherein an attachment collar is around the culture enrichment subassembly, and wherein the microfluidic cartridge subassembly comprises a channel manifold comprising one or more channels in fluidic communication with the culture enrichment chamber.

Example 2 is a microbiologic analysis assembly as in any of the examples, in particular example 1, wherein the microfluidic cartridge subassembly comprises a receptacle for attachment of the culture enrichment chamber subassembly and the channel manifold extending from the receptacle, wherein a rotatable selector valve assembly is in contact with the channel manifold.

Example 3 is a microbiological analysis assembly as in any of the examples, in particular example 2, wherein the platform comprises an upper panel, wherein the upper panel comprises an opening, wherein a rotatable shaft extends through the opening and is coupled to a slotted cap.

Example 4 is a microbiological analysis assembly as in any of the examples, in particular example 3, wherein the rotatable shaft comprises a cavity in fluidic communication with the opening, and wherein a plunger is within the cavity.

Example 5 is a microbiological analysis assembly as in a any of the examples, in particular example 4, wherein the rotatable selection valve is in abutted against the channel manifold, and wherein the opening in the rotatable selection valve is configured to align with one or more termini of one or more channels on the channel manifold.

Example 6 is a microbiological analysis assembly as in any of the examples, in particular example 5, wherein the one or more channels are in fluidic communication with one or more analysis chambers that extend away from the channel manifold, wherein the one or more analysis chambers are integral with the channel manifold.

Example 7 is a microbiological analysis assembly as in any of the examples, in particular example 6, the compliant insert comprises a compliant sac, wherein the compliant sac comprises a lip around a first rim of the compliant sac.

Example 8 is a microbiological analysis assembly as in any of the examples, in particular example 7, wherein the lip is configured to align under a second rim of a socket when the socket is seated within a base that is attached to a receptacle of the microfluidic cartridge subassembly.

Example 9 is a microbiological analysis assembly as in any of the examples, in particular example 8, wherein the receptacle comprises one or more cutting tabs having a knife edge, wherein the one or more cutting tabs are disposed on a rim of a culture termination chamber disposed on a floor of the receptacle.

Example 10 is an analysis station, comprising a mounting column affixed to a chassis; a stage affixed to the chassis; one or more ramps on the stage, wherein the one or more ramps comprise a ramped sidewall and a top sidewall, wherein one or more openings are within the top sidewall; and a heating block within the one or more ramps, wherein the heating block comprises at least one chamber in communication with the one or more openings.

Example 11 is an analysis station as in any of the examples, in particular example 10, wherein the stage is a first stage, and wherein the analysis station further comprises a second stage coupled to the chassis.

Example 12 is an analysis station as in any of the examples, in particular example 10, further comprising a vibrating plate adjacent to the heating block.

Example 13 is an analysis station as in any of the examples, in particular example 10, further comprising at least one circuit board affixed to the chassis, wherein the at least one circuit board comprises a processor.

Example 14 is an analysis station as in any of the examples, in particular example 13, wherein the at least one circuit board comprises sensors, motor drivers and power devices electrically coupled to the processor.

Example 15 is a method for operating an analysis station, comprising preparing a culture enrichment chamber subassembly; adding a microbiological sample into the culture enrichment chamber subassembly; attaching the culture enrichment subassembly to a microfluidic cartridge subassembly to construct a self-contained microbial analysis module (SCMAM) assembly; docking the SCMAM assembly to the analysis station; and performing an microbiological assay.

Example 16 is a method as in any of the examples, in particular example 15, wherein adding the microbiological sample into the culture enrichment chamber subassembly comprises inoculating the microbiological sample into a liquid growth medium contained within the compliant insert.

Example 17 is a method as in any of the examples, in particular example 15, wherein docking the SCMAM assembly to the analysis station comprises aligning one or more analysis chambers extending below the microfluidic cartridge by sliding the one or more analysis chambers up one or more ramps.

Example 18 is a method as in any of the examples, in particular example 15, wherein docking the SCMAM assembly to the analysis station comprises engaging one or more analysis chambers extending below the microfluidic cartridge with openings in one or more analysis chambers.

Example 19 is a method as in any of the examples, in particular example 15, wherein preparing the culture enrichment chamber subassembly comprises inserting a culture enrichment chamber into an attachment collar, assembling the attachment collar to a socket, and attaching the socket to a base.

Example 20 is a method as in any of the examples, in particular example 19, wherein assembling the attachment collar to the socket and attaching the socket to the base comprises rotating the attachment collar relative to the socket such that one or more first locking tabs fall into a first detent, and rotating the socket relative to the base such that one or more second locking tabs fall into a second detent.

Example 21 is a method as in any of the examples, in particular example 20,wherein attaching the culture enrichment subassembly to the microfluidic cartridge subassembly to construct the self-contained microbial analysis module (SCMAM) assembly comprises press-fitting the base into a receptacle portion of the microfluidic cartridge subassembly.

Example 22 is a microbiological analysis module comprising a culture enrichment chamber subassembly comprising a culture enrichment chamber insertable within an attachment collar, wherein the attachment collar is attachable to a cartridge adapter; and a microfluidic cartridge subassembly comprising a stage extending laterally from a receptacle, wherein the cartridge adapter of the culture enrichment chamber subassembly is attachable to the receptacle, wherein the stage comprises a channel manifold comprising one or more channels fluidically coupled to the culture enrichment chamber and to one or more analysis chambers, a prime chamber and one or more reagent chambers, wherein the one or more analysis chambers, the prime chamber and the one or more reagent chambers are integral with the channel manifold;

Example 23 is a microbial analysis module as in any of the examples, particularly example 22, further comprising a culture termination chamber within a cavity of the receptacle, wherein the culture termination chamber is configured to hold an antimicrobial substance; a movable plug within the culture termination chamber; and an access hole is disposed at a floor of the culture termination chamber, wherein the access hole is configured for a stylus to enter the culture termination chamber and push the movable plug into a cavity of the receptacle such that the antimicrobial substance is able to be released into the cavity of the receptacle.

Example 24 is a microbiological analysis module as in any of the examples, in particular example 22, wherein the stage comprises a midplane panel disposed over the channel manifold and an upper panel disposed over the midplane panel, and a sidewall surrounding the upper panel, the midplane panel and the channel manifold, and wherein a rotatable selector valve is in contact with the channel manifold, wherein the rotatable selector valve is part of a rotatable selector valve assembly comprising a shaft coupled to the rotatable selector valve, and wherein the shaft extends through the midplane panel to the upper panel, and wherein a slotted cap is attached to the shaft and extends above the upper panel.

Example 25 is a microbiological analysis module as in any of the examples, in particular example 24, wherein a hole in the upper panel is disposed over the prime chamber, and wherein the prime chamber is configured to receive a sample aliquot of an enrichment culture fluid from the culture enrichment chamber via a fluid transfer through the channel manifold, and the hole is configured to receive a syringe needle extending into the prime chamber to withdraw a sample of the enrichment culture fluid contained within the prime chamber.

Example 26 is a microbiological analysis module as in any of the examples, in particular example 24, wherein the shaft comprises a cavity fluidically coupled to an opening in the rotatable selector valve, and wherein a plunger is within the cavity, and wherein the plunger is configured to couple to an external vertically extensible shaft.

Example 27 is a microbiological analysis module as in any of the examples, in particular example 26, wherein an overflow chamber is adjacent to the one or more termini on the channel manifold, wherein the overflow chamber extends below a lower surface of the channel manifold.

Example 28 is a microbiological analysis module as in any of the examples, in particular example 23, wherein the culture termination chamber comprises one or more cutting tabs extending vertically above a rim of the culture termination chamber, wherein top sides of the one or more cutting tabs have a cutting edge, wherein the one or more cutting tabs are configured to cut open a sealing membrane disposed on the culture enrichment chamber subassembly, such that a culture enrichment fluid within the culture enrichment chamber is able to enter the cavity of the receptacle.

Example 29 is a microbiological analysis module as in any of the examples, in particular example 22, wherein the culture enrichment chamber comprises a compliant wall, wherein the compliant wall is configured to expand and contract such that the culture enrichment chamber is configured not to leak from internal pressurization of the culture enrichment chamber while under a hermetic seal, and wherein the culture enrichment chamber comprises a lip around a mouth of the culture enrichment chamber, wherein the lip is configured to form a hermetic seal by torque force against a gasket within the cartridge adapter.

Example 30 is a microbiological analysis module as in any of the examples, in particular example 22,, wherein the attachment collar is configured to rotatably fasten to a socket, wherein the socket comprises one or more first locking tabs and the attachment collar comprises one or more first detents configured to retain the one or more first locking tabs such that the attachment collar is rotatable by a first restricted angle of rotation.

Example 31 is a microbiological analysis module as in any of the examples, in particular example 30, wherein the socket is configured to rotatably fasten to the cartridge adapter, wherein the cartridge adapter comprises one or more second locking tabs and the socket comprises one or more second detents configured to retain the one or more second locking tabs such that the cartridge adapter is rotatable by a second angle of rotation.

Example 32 is a microbiological analysis module as in any of the examples, in particular example 22, wherein the one or more analysis chambers is configured to receive a light guide assembly comprising one or more light guide rods integral with an optical window, wherein the one or more light guide rods are disposed within the one or more analysis chambers, wherein the light guide assembly comprises one or more sealing rings at a top of the one or more light guide rods, wherein the one or more sealing rings form a hermetic seal with the one or more analysis chambers, and wherein the optical window is disposed over the one or more analysis chambers, and wherein the light guide assembly is configured to guide light into and out of the one or more analysis chambers.

Example 33 is an analysis station, comprising a mounting column affixed to a chassis, wherein a platform affixed to the chassis near the mounting column, wherein the platform is configured to seat a microbiological analysis module, wherein the microbiological analysis module comprises a culture enrichment chamber subassembly comprising a culture enrichment chamber insertable within an attachment collar, wherein the attachment collar is attachable to a cartridge adapter; and a microfluidic cartridge subassembly comprising a stage extending laterally from a receptacle, wherein the cartridge adapter of the culture enrichment chamber subassembly is attachable to the receptacle, wherein the stage comprises a channel manifold comprising one or more channels fluidically coupled to the culture enrichment chamber and to one or more analysis chambers, a prime chamber and one or more reagent chambers, wherein the one or more analysis chambers, the prime chamber and the one or more reagent chambers are integral with the channel manifold; one or more motors affixed on the mounting column, wherein the one or more motors have a shaft configured to rotate to a rotatable selector valve assembly within the platform of the microfluidic cartridge subassembly; and at least one heater block adjacent to the stage, wherein the at least one heater block comprises a cavity configured to receive the one or more analysis chambers.

Example 34 is an analysis station as in any of the examples, in particular example 33, wherein the stage comprises an opening configured to be aligned to an access hole at a bottom of the receptacle when the analysis station is seated on the stage, wherein the access hole is at the bottom of a culture termination chamber within the receptacle, wherein the access hole is configured to receive a stylus for entering the culture termination chamber and push a movable plug into the cavity of the receptacle such that an antimicrobial substance is able to be released into the cavity of the receptacle.

Example 35 is an analysis station as in any of the examples, in particular example 33, wherein the at least one heater block comprises one or more openings configured to provide access to light from one or more light sources on the mounting column to the one or more analysis chambers seated in the cavity of the at least one heater block.

Example 36 is an analysis station as in any of the examples, in particular example 33, further comprising an enclosure containing the mounting column and the chassis, wherein the enclosure is configured as to maintain a refrigeration temperature, and wherein a refrigerated extension within the enclosure is configured to thermally couple to the prime chamber and maintain the prime chamber at the refrigeration temperature.

Example 37 is a method for operating a microbiological analysis station, comprising preparing a culture enrichment chamber subassembly; adding a microbiological sample into a culture enrichment fluid contained within the culture enrichment chamber subassembly; attaching the culture enrichment chamber subassembly to a microfluidic cartridge subassembly to assemble a microbiological analysis module; docking the microbiological analysis module to the analysis station; performing a microbiological assay; and releasing an antimicrobial substance into the culture enrichment fluid.

Example 38 is a method as in any of the examples, in particular example 37,

wherein docking the microbiological analysis module to the analysis station comprises seating a receptacle portion of the microfluidic cartridge subassembly on a stage of the analysis station and inserting one or more analysis chambers extending below the microfluidic cartridge subassembly into openings in one or more heater blocks of the analysis station.

Example 39 is a method as in any of the examples, in particular example 37, wherein performing the microbiological assay comprises operating a rotatable selector valve within the microfluidic cartridge subassembly to draw an aliquot of solution into a first analysis chamber; operating the rotatable selector valve to draw a sample of the culture enrichment fluid containing a microbe culture into the first analysis chamber; causing a lysis of a plurality of microbes of the microbe culture in the first analysis chamber to release a plurality of microbial nucleic acid fragments into the solution; operating the rotatable selector valve to transfer a second aliquot of the solution containing the plurality of microbial nucleic acid fragments to a second analysis chamber; performing a reaction to amplify the plurality of microbial nucleic acid fragments, wherein a plurality of strands of amplified microbial nucleic acid is detected by exciting a fluorescence of a fluorophore attached to the plurality of strands of amplified microbial nucleic acid, by a bioluminescence, turbidity or a colorimetric signal.

Example 40 is a method as in any of the examples, in particular example 39, wherein operating the rotatable selector valve within the microfluidic cartridge subassembly to draw the aliquot of the solution into the first analysis chamber, wherein a solid substance is within the first analysis chamber, comprises shining a light of a third wavelength into the first analysis chamber and detecting the light of the third wavelength through a second light guide within the first analysis chamber, wherein the second light guide guides the light of the third wavelength to a photodetector is positioned under the first analysis chamber to receive the light of the third wavelength through the optical window.

Example 41 is a method as in any of the examples, in particular example 40, wherein releasing the antimicrobial substance into the culture enrichment fluid comprises extending a stylus into an access hole of a culture termination chamber within a receptacle portion of the microfluidic cartridge subassembly and pushing a plug out of the culture termination chamber such that the antimicrobial substance is released into the culture enrichment fluid to destroy the plurality of microbes within the culture enrichment fluid.

Example 42 is a method as in any of the examples, in particular example 39, wherein performing the microbiological assay comprises maintaining a solid substance at the bottom of a analysis chamber wherein the solid substance is held by the first or second light guide extending within the analysis chamber, wherein the solid substance remains within a fluid path of a liquid added to the analysis chamber.

Besides what is described herein, various modifications may be made to the disclosed embodiments and implementations thereof without departing from their scope. Therefore, illustrations of embodiments herein should be construed as examples only, and not restrictive to the scope of the present disclosure. The scope of the invention should be measured solely by reference to the claims that follow.

We claim:

1. A microbiological analysis module, comprising:
a culture enrichment chamber subassembly comprising a culture enrichment chamber insertable within an attachment collar, wherein the attachment collar is attachable to a cartridge adapter; and
a microfluidic cartridge subassembly comprising a stage extending laterally from a receptacle, wherein the cartridge adapter of the culture enrichment chamber subassembly is attachable to the receptacle, wherein the stage comprises a channel manifold comprising one or more channels fluidically coupled to the culture enrichment chamber and to one or more analysis chambers, a prime chamber and one or more reagent chambers, and wherein the one or more analysis chambers, the prime chamber and the one or more reagent chambers are integral with the channel manifold.

2. The microbiological analysis module of claim 1, further comprising:
a culture termination chamber within a cavity of the receptacle, wherein the culture termination chamber is configured to hold an antimicrobial substance;
a movable plug within the culture termination chamber; and
an access hole is disposed at a floor of the culture termination chamber, wherein the access hole is configured for a stylus to enter the culture termination chamber and push the movable plug into a cavity of the receptacle such that the antimicrobial substance can be released into the cavity of the receptacle.

3. The microbiological analysis module of claim 1, wherein the stage comprises a midplane panel disposed over the channel manifold and an upper panel disposed over the midplane panel, and a sidewall surrounding the upper panel, the midplane panel and the channel manifold, and wherein a rotatable selector valve is in contact with the channel manifold, wherein the rotatable selector valve is part of a rotatable selector valve assembly comprising a shaft coupled to the rotatable selector valve, and wherein the shaft extends through the midplane panel to the upper panel, and wherein a slotted cap is attached to the shaft and extends above the upper panel.

4. The microbiological analysis module of claim 3, wherein a hole in the upper panel is disposed over the prime chamber, and wherein the prime chamber is configured to receive a sample aliquot of an enrichment culture fluid from the culture enrichment chamber via a fluid transfer through the channel manifold, and the hole is configured to receive a syringe needle extending into the prime chamber to withdraw a sample of the enrichment culture fluid contained within the prime chamber.

5. The microbiological analysis module of claim 3, wherein the shaft comprises a cavity fluidically coupled to an opening in the rotatable selector valve, and wherein a plunger is within the cavity, and wherein the plunger is configured to couple to an external vertically extensible shaft.

6. The microbiological analysis module of claim 5, wherein an overflow chamber is adjacent to one or more termini on the channel manifold, and wherein the overflow chamber extends below a lower surface of the channel manifold.

7. The microbiological analysis module of claim 2, wherein the culture termination chamber comprises one or more cutting tabs extending vertically above a rim of the culture termination chamber, wherein top sides of the one or more cutting tabs have a cutting edge, and wherein the one or more cutting tabs are configured to cut open a sealing membrane disposed on the culture enrichment chamber subassembly, such that a culture enrichment fluid within the culture enrichment chamber is able to enter the cavity of the receptacle.

8. The microbiological analysis module of claim 1, wherein the culture enrichment chamber comprises a compliant wall, wherein the compliant wall is configured to expand and contract such that the culture enrichment chamber is configured not to leak from internal pressurization of the culture enrichment chamber while under a hermetic seal, wherein the culture enrichment chamber comprises a lip around a mouth of the culture enrichment chamber, and wherein the lip is configured to form a hermetic seal by torque force against a gasket within the cartridge adapter.

9. The microbiological analysis module of claim 1, wherein the attachment collar is configured to rotatably fasten to a socket, wherein the socket comprises one or more first locking tabs and the attachment collar comprises one or more first detents configured to retain the one or more first locking tabs such that the attachment collar is rotatable by a first restricted angle of rotation.

10. The microbiological analysis module of claim 9, wherein the socket is configured to rotatably fasten to the cartridge adapter, and wherein the cartridge adapter comprises one or more second locking tabs and the socket comprises one or more second detents configured to retain the one or more second locking tabs such that the cartridge adapter is rotatable by a second angle of rotation.

11. The microbiological analysis module of claim 1, wherein the one or more analysis chambers is configured to receive a light guide assembly comprising one or more light guide rods integral with an optical window, wherein the one or more light guide rods are disposed within the one or more analysis chambers, wherein the light guide assembly comprises one or more sealing rings at a top of the one or more light guide rods, wherein the one or more sealing rings form a hermetic seal with the one or more analysis chambers, wherein the optical window is disposed over the one or more analysis chambers, and wherein the light guide assembly is configured to guide light into and out of the one or more analysis chambers.

12. An analysis station, comprising:
a mounting column affixed to a chassis; and
a platform affixed to the chassis near the mounting column, wherein the platform is configured to seat a microbiological analysis module, wherein the microbiological analysis module comprises:
a culture enrichment chamber subassembly comprising a culture enrichment chamber insertable within an attachment collar, wherein the attachment collar is attachable to a cartridge adapter;
a microfluidic cartridge subassembly comprising a stage extending laterally from a receptacle, wherein the cartridge adapter of the culture enrichment chamber subassembly is attachable to the receptacle, wherein the stage comprises a channel manifold comprising one or more channels fluidically coupled to the culture enrichment chamber and to one or more analysis chambers, a prime chamber and one or more reagent chambers, wherein the one or more analysis chambers, the prime chamber and the one or more reagent chambers are integral with the channel manifold;
one or more motors affixed on the mounting column, wherein the one or more motors have a shaft configured to rotate to a rotatable selector valve assembly within the platform of the microfluidic cartridge subassembly; and
at least one heater block adjacent to the stage, wherein the at least one heater block comprises a cavity configured to receive the one or more analysis chambers.

13. The analysis station of claim 12, wherein the stage comprises an opening configured to be aligned to an access hole at a bottom of the receptacle when the analysis station is seated on the stage, wherein the access hole is at the bottom of a culture termination chamber within the receptacle, wherein the access hole is configured to receive a stylus for entering the culture termination chamber and push a movable plug into the cavity of the receptacle such that an antimicrobial substance is able to be released into the cavity of the receptacle.

14. The analysis station of claim 12, wherein the at least one heater block comprises one or more openings configured to provide access to light from one or more light sources on the mounting column to the one or more analysis chambers seated in the cavity of the at least one heater block.

15. The analysis station of claim 12, further comprising an enclosure containing the mounting column and the chassis, wherein the enclosure is configured as to maintain a refrigeration temperature, and wherein a refrigerated extension within the enclosure is configured to thermally couple to the prime chamber and maintain the prime chamber at the refrigeration temperature.

16. A method for operating a microbiological analysis station, comprising;
preparing a culture enrichment chamber subassembly;
adding a microbiological sample into a culture enrichment fluid contained within the culture enrichment chamber subassembly;
attaching the culture enrichment chamber subassembly to a microfluidic cartridge subassembly to assemble a microbiological analysis module;
docking the microbiological analysis module to the microbiological analysis station;
performing a microbiological assay; and
releasing an antimicrobial substance into the culture enrichment fluid.

17. The method of claim 16, wherein docking the microbiological analysis module to the microbiological analysis station comprises seating a receptacle portion of the microfluidic cartridge subassembly on a stage of the microbiological analysis station and inserting one or more analysis chambers extending below the microfluidic cartridge subassembly into openings in one or more heater blocks of the microbiological analysis station.

18. The method of claim 16, wherein performing the microbiological assay comprises:
operating a rotatable selector valve within the microfluidic cartridge subassembly to draw an aliquot of solution into a first analysis chamber;
operating the rotatable selector valve to draw a sample of the culture enrichment fluid containing a microbe culture into the first analysis chamber;
causing a lysis of a plurality of microbes of the microbe culture in the first analysis chamber to release a plurality of microbial nucleic acid fragments into the solution;
operating the rotatable selector valve to transfer a second aliquot of the solution containing the plurality of microbial nucleic acid fragments to a second analysis chamber; and
performing a reaction to amplify the plurality of microbial nucleic acid fragments, wherein a plurality of strands of amplified microbial nucleic acid are detected by exciting a fluorescence of a fluorophore attached to the plurality of strands of amplified microbial nucleic acid, by a bioluminescence, turbidity or a colorimetric signal.

19. The method of claim 18, wherein operating the rotatable selector valve within the microfluidic cartridge subassembly to draw the aliquot of the solution into the first analysis chamber, wherein a solid substance is within the first analysis chamber, comprises shining a light into the first analysis chamber and detecting the light through a light guide within the first analysis chamber, and wherein the light guide guides the light to a photodetector is positioned under the first analysis chamber to receive the light through an optical window.

20. The method of claim 19, wherein releasing the antimicrobial substance into the culture enrichment fluid comprises extending a stylus into an access hole of a culture termination chamber within a receptacle portion of the microfluidic cartridge subassembly and pushing a plug out of the culture termination chamber such that the antimicrobial substance is released into the culture enrichment fluid to destroy the plurality of microbes within the culture enrichment fluid.

21. The method of claim 19, wherein performing the microbiological assay comprises maintaining a solid substance at a bottom of an analysis chamber wherein the solid substance is held under the light guide extending within the analysis chamber, and wherein the solid substance remains within a fluid path of a liquid added to the analysis chamber.
